SEQUENCE LISTING (54 sequences)

SEQ ID NO: 1 - DNA, 27 bp, artificial
  chromosome 12 1-FW outer primer MDM2
ataggactgaggtaattctgcacagca

SEQ ID NO: 2 - DNA, 26 bp, artificial
  Chromosome 12 2-Rev-inner primer MDM2
ataatgcttggaggacctccacatgt

SEQ ID NO: 3 - DNA, 27 bp, artificial
  Chromosome 12 3-FW-inner primer MDM2
taaatggccaaagggattagtagtgtg

SEQ ID NO: 4 - DNA, 28 bp, artificial
  chromosome 12 4-Rev-Outer primer MDM2
aagagattctgcttggttgtagctgaag

SEQ ID NO: 5 - DNA, 28 bp, artificial
  chromosome 22 1-FW-Outer primer APOBEC3D
ctctgggatctctctgcctccaaatatc

SEQ ID NO: 6 - DNA, 24 bp, artificial
  chromosome 22 2-Rev-inner primer APOBEC3D
gaggttgcagtgagtccagatggc

SEQ ID NO: 7 - DNA, 23 bp, artificial
  chromosome 22 3-FW-inner primer APOBEC3D
gtccaggctggaatgcaatgtca

SEQ ID NO: 8 - DNA, 28 bp, artificial
  chromosome 22 APOBEC3D 4-Rev-Outer primer
gaggctgaagcagaagaatcgcttaaac

SEQ ID NO: 9 - DNA, 29 bp, artificial
  chromosome 22 1-FW-Outer primer APOBEC3D
tttgagacagagtgttgctcctcttgtcc

SEQ ID NO: 10 - DNA, 26 bp, artificial
  chromosome 22 3-FW-inner primer APOBEC3D
cgggaggctgaagcagaagaatctct

SEQ ID NO: 11 - DNA, 22 bp, artificial
  chromosome 22 3-FW-inner primer APOBEC3D
aacctccgcctcccgagttgag

SEQ ID NO: 12 - DNA, 28 bp, artificial
  chromosome 22 4-Rev-Outer primer APOBEC3D
caaaattaaccaggtgtggtgatgcatg

SEQ ID NO: 13 - DNA, 29 bp, artificial
  chromosome 1 1-FW-Outer primer SRP9
cactgtctcaaaaatacataccttcagca

SEQ ID NO: 14 - DNA, 22 bp, artificial
  chromosome 1 2-Rev-inner primer SRP9
cttgaacccaggagtgaggtcc

SEQ ID NO: 15 - DNA, 22 bp, artificial
  chromosome 1 3-FW-inner primer SRP9
ggcatgatcttggctcacttca

SEQ ID NO: 16 - DNA, 28 bp, artificial
  chromosome 1 4-Rev-Outer primer SRP9
ctaaaaatacaaaaattagctgggcgtg

SEQ ID NO: 17 - DNA, 28 bp, artificial
  chromosome 12 1-FW-Outer primer Gli 1
ggggaggacagaactttgatccttacct

SEQ ID NO: 18 - DNA, 22 bp, artificial
  chromosome 12 2-Rev-inner primer Gli 1
ctggctcttcctgtagcccgct

SEQ ID NO: 19 - DNA, 25 bp, artificial
  chromosome 12 3-FW-inner primer Gli 1r
actgagaatgctgccatggatgatg

SEQ ID NO: 20 - DNA, 28 bp, artificial
  chromosome 12 Gli 1 4-Rev Outer primer
aagtccatataggggttcagaccactgc

SEQ ID NO: 21 - DNA, 26 bp, artificial
  chromosome 12 Gli (neg ctrl) 1-FW-Outer primer
atatcctgaccccacccaagaaacat

SEQ ID NO: 22 - DNA, 25 bp, artificial
  chromosome 12 Gli 1(neg ctrl) 2-Rev-inner primer
aggacactggctgtaggttccaacc

SEQ ID NO: 23 - DNA, 23 bp, artificial
  chromosome 12 Gil 1 (neg ctrl) 3-FW-inner primer
tgtacccaggccccaaggctata

SEQ ID NO: 24 - DNA, 28 bp, artificial
  chromsome 12 Gli 1 (neg ctrl) 4-Rev-Outer primer
ggcttgacttgcacttgtccataatgtt

SEQ ID NO: 25 - DNA, 28 bp, artificial
  chromosome 25 GSK3B 1-FW-Outer primer
caaaggcaagtctataaatacccgaaga

SEQ ID NO: 26 - DNA, 32 bp, artificial
  chromosome 3 GSK3B 2-Rev-inner primer
tcttttaaagtctagtgtgagactttggtctg

SEQ ID NO: 27 - DNA, 22 bp, artificial
  chromosome 3 GSK3B 3-FW-inner primer
aaccaatttcaagctgtgccct

SEQ ID NO: 28 - DNA, 28 bp, artificial
  chromosome 3 GSK3B 4-Rev-Outer primer
aagaagaaaacctttttctgtgctgatg

SEQ ID NO: 29 - DNA, 22 bp, artificial
  chromosome 8 AZIN1 1-FW-Outer primer
tttcccaagctgaaaagacaag

SEQ ID NO: 30 - DNA, 22 bp, artificial
  chromosome 8 AZIN1 2-REV-innerr primer
gagcttgatcaaattgtggcag

SEQ ID NO: 31 - DNA, 25 bp, artificial
  chromosome 8 AZIN1 3-FW-inner primer
cattcagctcaggaagaagacatct

SEQ ID NO: 32 - DNA, 28 bp, artificial
  chromosome 8 AZIN1 4-Rev-Outer primer
aatacaaggaagatgagcctctgtttac

SEQ ID NO: 33 - DNA, 22 bp, artificial
  chromosome 1 PTPN14 1-FW-Outer primer
tttcccaagctgaaaagacaag

SEQ ID NO: 34 - DNA, 25 bp, artificial
  chromosome 1 PTPN14 2-Rev-inner primer
ccttttcttgagtacagctttgcta

SEQ ID NO: 35 - DNA, 22 bp, artificial
  chromosome 1 PTPN14 3-FW-inner primer
caccacgtaacaacaggtgaac

SEQ ID NO: 36 - DNA, 28 bp, artificial
  chromosome 1 PTPN14 4-Rev-outer primer
ttttgtacttttttcctcttactgcatt

SEQ ID NO: 37 - DNA, 26 bp, artificial
  chromosome 16 SF3B3 1-FW-Outer primer
tcacacctgtaatcccagcactttga

SEQ ID NO: 38 - DNA, 22 bp, artificial
  chromosome 16 SF3B3 2-Rev-inner primer
gtgtgcaccaccatgcctgtct

SEQ ID NO: 39 - DNA, 30 bp, artificial
  chromosome 16 SF3B3 3-FW-inner primer
aaccccatctctccaaaaatacaaaaagtg

SEQ ID NO: 40 - DNA, 28 bp, artificial
  chromosome 16 SF3B3 4-Rev-outer primer
ataggctgaagtgatcctcctgtctcag

SEQ ID NO: 41 - DNA, 28 bp, artificial
  chromosome 10 ABI1 1-FW-Outer primer
agagagccaagaaataagcctttaaggg

SEQ ID NO: 42 - DNA, 28 bp, artificial
  Chromosome 10 ABI1 2-Rev-inner prime
tatttttagtagacacgggttttcgacg

SEQ ID NO: 43 - DNA, 23 bp, artificial
  chromosome 10 ABI1 3-FW-inner primer
tttgagaccagcctggccataat

SEQ ID NO: 44 - DNA, 28 bp, artificial
  chromosome 10 ABI1 4-Rev-outer primer
gttcaagtgattctcctgtctcaacctc

SEQ ID NO: 45 - DNA, 29 bp, artificial
  chromosome 1 LYST 1-FW-outer primer
tggcaaaaccctgtttctactaaaaatat

SEQ ID NO: 46 - DNA, 23 bp, artificial
  chromosome 1 LYST 2-Rev-inner primer
gttctagaggttgtcatgtcgcg

SEQ ID NO: 47 - DNA, 26 bp, artificial
  chromosome 1 LYST 3-FW-inner primer
ctgtaatcccagctactcaggtgtct

SEQ ID NO: 48 - DNA, 27 bp, artificial
  chromosome 1 LYST 4-Rev-outer primer
tttgagacagagtctcactttttcacc

SEQ ID NO: 49 - DNA, 28 bp, artificial
  chromosome 1 MDM4 1-FW-Outer primer
tggaagaagaattgtcttggaccacaca

SEQ ID NO: 50 - DNA, 28 bp, artificial
  chromosome 1 MDM4 2-Rev-inner primer
ctaggtgatctcccaaagtgttgggctt

SEQ ID NO: 51 - DNA, 22 bp, artificial
  chromosome 1 MDM4 3-FW-inner primer
ggcgtagtggctcacgcctttg

SEQ ID NO: 52 - DNA, 28 bp, artificial
  chromosome 1 MDM4 4-Rev-Outer primer
gtaaaaatggggtttctccatgtcggtc

SEQ ID NO: 53 - DNA, 33 bp, artificial
  primer for generating ADAR1 mutation
gtcaatgactgccatgcagcaataatctcccgg

SEQ ID NO: 54 - DNA, 33 bp, artificial
  primer for generating ADAR1 mutation
ccgggagattattgctgcatggcagtcattgac

(19) United States
(12) Patent Application Publication
(10) Pub. No.: US 2017/0191057 A1
(43) Pub. Date: Jul. 6, 2017

(54) RNA EDITING BIOMARKERS FOR DIAGNOSIS, PHARMACOLOGICAL SCREENING AND PROGNOSTICATION IN CANCER

(71) Applicant: REGENTS OF THE UNIVERSITY OF CALIFORNIA, Oakland, CA (US)

(72) Inventors: Catriona H. Jamieson, La Jolla, CA (US); Qingfei Jiang, La Jolla, CA (US); Leslie Crews Robertson, La Jolla, CA (US); Maria Anna Zipeto, La Jolla, CA (US); Anil Sadarangani, La Jolla, CA (US); Gabriel Pineda, La Jolla, CA (US); Kathleen M. Kane, La Jolla, CA (US)

(21) Appl. No.: 15/117,038

(22) PCT Filed: Feb. 5, 2015
(86) PCT No.: PCT/US15/14686

§ 371 Date: Aug. 5, 2016

Related U.S. Application Data
(60) Provisional application No. 61/936,314, filed on Feb. 5, 2014.

(51) Int. Cl. C12N 15/113; C12N 5/095; A61K 31/7064; C12Q 1/68

(52) U.S. Cl. CPC C12N 15/113; C12Q 1/6886; C12Q 1/6897; C12N 5/0695; A61K 31/7064; C12Q 2600/118; C12Q 2600/158; C12Q 2600/136; C12Q 2600/178; C12N 2310/113; C12N 2310/14; C12N 2310/12; C12N 2310/531; C12N 2510/00

(57) ABSTRACT

Compositions and methods for expanding CD34+ cells, performing research related to cancer stem cells, RNA-editing enzymes and for monitoring, diagnosing and treating, ameliorating and preventing diseases such as cancers or inflammatory diseases.

### FIELD OF THE INVENTION

The present disclosure relates to the field of oncology, biomarkers, biology and drug discovery. Particularly, the disclosure relates to compositions useful for studying the cell cycle, RNA-editing enzymes, monitoring of disease progression, and pharmacological screening. The disclosure also includes methods for expanding stem cells. The disclosure also relates to compositions and methods for inhibiting the action of double-stranded RNA-specific adenosine deaminases, or ADAR enzymes. The disclosure includes methods and compositions for treating, ameliorating or preventing diseases and conditions, such as cancer, including cancers associated with stem cells such as, without limitation, a myeloproliferative neoplasm like chronic myeloid leukemia (CML) or acute myeloid leukemia (AML).

### BACKGROUND OF THE DISCLOSURE

RNA editing is a post-transcriptional processing mechanism that results in an RNA sequence that is different from that encoded by the genomic DNA and thereby diversifies the gene product and function. The type of RNA editing that is most prevalent in higher eukaryotes converts adenosine residues into inosine (A-to-I editing) in double-stranded RNA (dsRNA) through the action of double-stranded RNA-specific adenosine deaminases (Bass B L, Weintraub H, Cell, 1988; 55(6):1089-98). RNA-specific adenosine deaminases include ADAR1 (also known as ADAR), ADAR2 (ADARB1), and ADAR3 (ADARB2). ADAR1 and ADAR2 are active in embryonic cell types, and ADAR3 may play a nonenzymatic regulatory role in RNA-editing activity (Osenberg S, et al., GPLoS One, 2010, 5(6):e11173; Chen C X et al., RNA, 2000, 6(5):755-767.

The ADAR1 enzyme destabilizes double-stranded RNA through conversion of adenosine to inosine. The ADAR1 enzyme modifies cellular and viral RNAs, including coding and noncoding RNAs. ADAR1 targets double-stranded RNA hairpin-containing loop structures, such as microRNAs (miRNAs) by operating through base-pairing with complementary sequences within an mRNA molecule leading to mRNA degradation and gene silencing. ADAR activity is suggested in various tumor types (Galeano F. et al., Semin Cell Dev Biol, 2012 23(3):244-250; Cenci et al., J Biol Chem, 2008, 283(11):7251-7260).

Traditional treatments for myeloproliferative neoplasm are a great financial burden on patients. Moreover, they are not efficient at eradicating cancer stem cells (CSC), such as leukemia cancer stem cells (LCS). Studies suggest that LCS promote therapeutic resistance, relapse and disease progression, the leading causes of leukemia mortality, as a result of enhanced survival and self-renewal combined with a propensity to become dormant in supportive microenvironments. Therapies capable of breaking LSC quiescence while sparing normal hematopoietic stem cell (HSC) function have remained elusive. New drugs that target cancer stem cells are urgently needed for patient care as well as research tools that aid in the studies of the role cancer stem cells play in cancer. The disclosure herein provides drugs and research tools useful in prognostication, pharmacological screening, treating and studying cancer.

### SUMMARY OF THE INVENTION

Disclosed herein are methods of expanding cells, such as stem cells and hematopoietic cells, comprising increasing ADAR1 activity in such cells, such as by overexpression of ADAR1 on a plasmid. In some aspects of this embodiment, the cells are cord blood cells (CB), such as cord blood cells that are CD34⁺. Included in this embodiment are the cell or cells that overexpress ADARI.

Another embodiment disclosed herein are methods for transplanting hematopoietic-reconstituting cells into a subject in need thereof, the method comprising administering to the subject CD34⁺ cells having enhanced expression of ADAR1. In an aspect of this embodiment, the subject has a disorder treatable by hematopoietic stem cell transplantation, such as a hematopoietic deficiency or malignancy.

In a related embodiment are methods for reconstituting hematopoietic cells into subjects in need thereof comprising: a) collecting cord blood cells from a donor or donors; b) transducing into the cells from step (a) a vector that overexpresses ADARI; c) expanding the transduced cells for about 3-5 days; d) collecting the cells from step (c); and e) transplanting the expanded cells from step (D) into the subject in need of treatment. In some aspects of this method the cord blood cells are CD34⁺. In some embodiments of this method the subject is being treated for leukemia, lymphoma or other blood-related diseases. In some aspects of this method the subjects blood supply has been damaged by chemotherapy, radiation, or toxic agent and/or the patient needs a bone marrow transplant. In some aspects of this method the cells to be expanded are transduced with a vector that overexpresses ADAR1 such as a lentiviral vector. In some embodiments of the method successful expansion is measured by an elevation in CD45⁺ as compared to the CD45⁺ levels in cord blood cells without overexpression of ADAR1 or a normal control. In some embodiments the expanded cells are CD34⁺ CD38⁻ and Lin⁻ human stem cells.

Also disclosed herein are vectors that overexpress ADAR1 and vectors that express a mutant version of ADAR1 protein wherein the mutation results in an ADAR1 protein that retains dsDNA and dsRNA binding capacity but has reduced ability to convert A-to-I activity. In aspects of this embodiment, the mutant ADAR1 protein has a mutation in the active RNA-editing sites, such as a point mutation, for example, a point mutation at nt5293 which results in an A to C mutation resulting in glutamic acid to alanine change. In some aspects the vector is a human-specifc lentivaral vector. Also included in this embodiment are cells containing the disclosed ADAR1 vectors. In some embodiments the cells are K562 cells.

Another embodiment disclosed herein is a reporter vector for measuring A-to-I editing comprising dual-luciferase, or enhanced green fluorescence protein (EGFP) or enhanced yellow fluorescence protein (EYFP) and a stop codon (TAG) in a hairpin structure which is part of the promoter sequence, wherein the stop codon is removed due to RNA editing resulting in a reporter gene signal being generated as a readout of RNA-editing level. In some aspects this vector contains an opposite oriented Alu-sequence to detect RNA editing in non-coding regions. Included in this embodiment are cells containing the vectors, such as stem cells and cancer stem cells. In an aspect of this embodiment are methods for measuring and tracing the A-to-I RNA-editing changes in a cancer stem cell comprising introducing the vectors into cancer stem cells and correlating changes in A-to-I RNA editing after exposure to agents. In some aspects the cancer stem cells are in in vitro stromal co-culture system or in vivo xenograft mouse models.

In another embodiment are vectors comprising wild-type GLI2 cloned into pcDH-EF1-T2A-cop and ligated in frame with the FLAG epitope. In a related embodiment are vectors comprising mutant GLI2 wherein the transcription activation domain has been deleted, In some embodiments the mutant GLI2 is cloned into pcDH-EF1-T2A in frame with the FLAG epitope. Included in these embodiments are cells containing the GLI2 wild-type and mutant GLI2 vectors. The cells can be stem cells, such as cancer stem cells.

Also disclosed herein are vectors comprising a bi-cistronic fluorescent ubiquitination-based cell-cycle indicator (FUCCI) reporter in a vector that is suitable for use in mammalian cell populations and which are useful for studying the cell cycle. In some embodiments the vector contains a bi-cistronic FUCCI reporter that was generated by cloning mCherryhCdt1 and Venus-hGeminin into vector pcDH-EF1-T2A. Included in this embodiment are cells that contain the bi-cistronic FUCCI reporter vectors. The cells can be stem cells, such as cancer stem cells.

In another embodiment are methods for treating, ameliorating or preventing diseases and conditions associated with the down regulation of one or more microRNA identified in FIGS. 8-11 comprising: administering to a subject in need of treatment a composition that upregulates one or more of the downregulated microRNA, wherein the composition is one or more of antisense DNA, RNAi, ribozyme, short hairpin RNA, a small molecule, an antibody or antibody fragment, a small HA oligosaccharide, or soluble HA-binding proteins. In some aspects the downregulation of the microRNA is associated with a stem cell, such as a cancer stem cell, for example a leukemia stem cell. In some aspects of this embodiment the condition or disease to be treated is cancer or an inflammatory disease, such as a myeloproliferative neoplasm, for example, chronic myeloid leukemia or acute myeloid leukemia. In some aspects of this embodiment, the chronic myeloid leukemia is in the blast phase. In still other aspects of this embodiment the composition administered upregulates Let7a. In some embodiments the upregulator of Let7a is a vitamin D3 derivative. In other embodiments, the patient is treated with an inhibitor or ADAR1, such as 8-azaadenosine or an 8-azaadenosine derivative.

As used herein, the term “subject” refers to an animal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); other mammals such as rodents (mice, rats), cattle, pigs, horses, sheep, goats, cats, dogs; and/or birds, that will be or has been the object of treatment, observation, and/or experiment. When the term is used in conjunction with administration of an, agent, composition, compound or drug, then the subject/patient has been the object of treatment, observation, and/or administration of the composition, compound or drug.

In an embodiment disclosed herein are methods for monitoring if a subject with cancer or an inflammatory disease should be treated or enrolled in a clinical trial comprising: (a) isolating blood cells from the subject during or after treatment; (b) processing the isolated stems cells to detect and/or quantitate one or more microRNA identified in FIGS. 8-11; (c) determining if the level of expression of the microRNA has decreased or increased from the levels of expression of the microRNA seen before treatment or as compared to normal controls; wherein if the level of expression of one or more microRNA has decreased and is a microRNA associated with a disease state, such as mir26a-sp, mir26b-5p, mir155-5p, mir21-5p, mir125a-5p, mir23b-3p, let7c, let7e, mir150-5p, and let7d the subject should be treated or enrolled in a clinical trial. In some aspects of this embodiment, the subject has been diagnosed with CML. In a related embodiment, the subject is treated with an agent/compound and the subject is then monitored to determine if the level of expression of a microRNA shown herein to be decreased in a disease state has increased from the levels of expression of the microRNA seen before treatment, wherein if the level of expression of one or more microRNA identified in FIGS. 8-11, such as mir26a-sp, mir26b-5p, mir155-5p, mir21-5p, mir125a-5p, mir23b-3p, let7c, let7e, mir150-5p is upregulated after treatment, such upregulation can be used as an indicator that the treatment is working.

In another embodiment disclosed herein are primers comprising, consisting of, or consisting essentially of the primers identified in FIG. 16 and Table 1.

Disclosed herein are methods for screening for agonists and inhibitors of RNA editing comprising: (a) contacting a CD34⁺ cell which overexpresses ADAR1 with a test compound; and (b) determining whether the test compound acts as an agonist or inhibitor. In some aspects the determination is measured using the fluorescent A-to-I editing reporter described in FIG. 3A which is transduced into the CD34⁺ cell. In other aspects the determination is done through use of RESSq-PCR or qPCR using direct sequencing of CSC-specific RNA-editing biomarkers or microRNAs as described in FIGS. 8-11, 14 and FIG. 16.

An embodiment disclosed here is a method of treating, ameliorating or preventing diseases and conditions responsive to the inhibition or slowing of cell differentiation and/or self-renewal (or self-renewal capacity) of dysfunctional cells, cancer cells, leukemia cells, hematopoietic stem cells or cancer stem cells, comprising, administering 8-azaadenosine or an 8-azaadenosine derivative. In some aspects the 8-azaadenosine derivative is a compound as shown in FIG. 25. In other aspects of this embodiment, the disease or condition is cancer or inflammatory disease, such as a myeloproliferative neoplasm, for example chronic myeloid leukemia (CML) or acute myeloid leukemia (AML).

In still other embodiments are methods for detecting leukemic progression into blast phase comprising the steps of: (a) collecting a blood sample from a subject with leukemia; (b) isolating mononuclear cells from the blood sample; (c) isolating CD34⁺ cells; (d) isolating RNA from the CD34⁺ cells; (e) generating cDNA from the isolated RNA; (f) performing quantitative PCR using one or more primer sets for the genes listed in Table 1—MDM2, APOBEC3D, GLI1, AZIN1, SRPN, GSK3B, PTPN14, SF3B3, ABI1, LYST; and (g) quantifying the data to determine the relative A-to-G(I) editing ratios in one or more of MDM2, APOBEC3D, GLI1, AZIN1, SRPN, GSK3B, PTPN14, SF3B3, ABI1, LYST and MDM4, wherein if the ratio of edited is higher than a normal standard or a previous determination from the same patient obtained in chronic phase such increased editing activity indicates the patient may be in blast phase or entering blast phase and should be treated or entered into a clinical trial. In some embodiments of this method, the one or more primer sets are represented by SEQ ID Nos. 1-4 for MDM2 position 69237534; SEQ ID NOS. 5-8 for APOBEC3D position 39415872; SEQ ID NOS:9-12 for APOBEC3D position 39415911; SEQ ID NOS:13-16 for SRP9 position 225976198; SEQ ID NOS: 17-20 for Gli1 position 57864624; SEQ ID NOS:21-24 for Gli1 position 57864911(negative control); SEQ ID NOS:25-28 for GSK3B position 119545199; SEQ ID NOS:29-32 for AZIN position 103841636; SEQ ID NOS:33-36 for PTPN14 position 214529774; SEQ ID NOS:37-40 for SF3B3 position 70610885; SEQ ID NOS:41-44 for ABI1 position 27049636; SEQ ID NOS:45-48 for LYST position 235990569; and SEQ ID NOS:49-52 for MDM4 position 204521159. In some embodiments of this method, the cDNA preparation utilizes gene transcription with reverse transcription and gene-specific primers and/or random hexamer primers only and/or a supermix containing both random hexamer and oligo-dT primers.

Disclosed herein are methods for testing whether an agent is effective for reducing the editing activity of ADAR1 comprising: (a) adding the agent to cells that have ADAR1 activity; (b) isolating RNA from the cells of step (a); (c) generating cDNA from the isolated RNA; (d) performing quantitative PCR using one or more primer sets for the genes listed in Table 1—MDM2, APOBEC3D, GLI1, AZIN1, SRPN, GSK3B, PTPN14, SF3B3, ABI1, LYST; and (e) quantifying the data to determine the relative A-to-G(I) editing ratios in one or more of MDM2, APOBEC3D, GLI1, AZIN1, SRPN, GSK3B, PTPN14, SF3B3, ABI1, LYST and MDM4, wherein if the ratio of edited/wild-type is lower than cells not treated with the tested agent indicates that the agent lowers the editing activity of ADAR1 and may be useful for treating conditions associated with increased levels of ADAR1 activity, such as leukemia and inflammatory diseases. In some embodiments of this method the one or more primer sets are represented by SEQ ID Nos. 1-4 for MDM2 position 69237534; SEQ ID NOS. 5-8 for APOBEC3D position 39415872; SEQ ID NOS:9-12 for APOBEC3D position 39415911; SEQ ID NOS:13-16 for SRP9 position 225976198; SEQ ID NOS: 17-20 for Gli1 position 57864624; SEQ ID NOS:21-24 for Gli1 position 57864911(negative control); SEQ ID NOS:25-28 for GSK3B position 119545199; SEQ ID NOS:29-32 for AZIN position 103841636; SEQ ID NOS:33-36 for PTPN14 position 214529774; SEQ ID NOS:37-40 for SF3B3 position 70610885; SEQ ID NOS:41-44 for ABI1 position 27049636; SEQ ID NOS:45-48 for LYST position 235990569; and SEQ ID NOS:49-52 for MDM4 position 204521159. In some aspects of this embodiment, the cells that have ADAR1 activity are cells containing a wild-type ADAR1 expression vector. In other aspects of the methods the cells are K562 cell. In some embodiments, the K562 cells are stably-transduced with lentiviral-ADAR1. In other aspects of this method the cells treated with an agent are CD34⁺ cells obtained from primary CML patients; or cells obtained from an immunocompromised non-human animal transplanted with primary blast crisis cells, such as, without limitation, a mouse.

In another embodiment are methods for detecting leukemic progression into blast phase comprising the steps of: (a) collecting a blood sample from a subject with leukemia; (b) isolating mononuclear cells from the blood sample; (c) isolating CD34⁺ cells; (d) isolating RNA from the CD34⁺ cells; (e) converting the RNA from step (d) into cDNA; (f) evaluating miRNA expression using MiScript qPCR array; and (g) determining if one or more of mir26a-5p, mir26b-5p, mir155-5P, mir21-5P, mir125a-5P, mir23b-3P, let7c, let7e, mir150-5p, or let7d are downregulated as compared to a normal control or a previous sample from the subject while in chronic phase, wherein if one or more of mir26a-5p, mir26b-5p, mir155-5P, mir21-5P, mir125a-5P, mir23b-3P, let7c, let7e, mir150-5p, or let7d are downregulated indicates that the patient is in or entering blast phase and should be treated or enrolled in a clinical trial.

Also disclosed herein are methods for testing whether an agent is effective for reducing the editing activity of ADAR1 comprising: (a) adding the agent to cells that have ADAR1 activity; (b) isolating RNA from the cells of step (a); (c) generating cDNA from the isolated RNA; (d) evaluating miRNA expression using MiScript qPCR array; (e) determining if one or more of mir26a-5p, mir26b-5p, mir155-5P, mir21-5P, mir125a-5P, mir23b-3P, let7c, let7e, mir150-5p, or let7d are upregulated as compared to cells not treated with the tested agent, wherein if one or more of mir26a-5p, mir26b-5p, mir155-5P, mir21-5P, mir125a-5P, mir23b-3P, let7c, let7e, mir150-5p, or let7d are upregulated this may indicate that the tested agent inhibits ADAR1 activity and may be useful to treat conditions associated with high ADAR1 editing activity, such as leukemia or inflammatory diseases. In some embodiments of this method, the cells that have ADAR1 activity are cells containing a wild-type ADAR1 expression vector. In other embodiments of this method, the cells are K562 cells. In other embodiments of this method the cells are K562 cells stably-transduced with lentiviral-ADAR1.

As used herein, the terms “compositions,” “drug,” “agent,” “compound,” and “therapeutic agent” are used interchangeably, and may include, without limitation, small molecule compounds, biologics (e.g., antibodies, proteins, protein fragments, fusion proteins, glycoproteins, etc.), nucleic acid agents (e.g., antisense, RNAi/siRNA, shRNA, and microRNA molecules, etc.), vaccines, etc., which may be used for therapeutic and/or preventive treatment of a disease (e.g., malignancy).

“Therapeutically effective amount,” or “therapeutic effect,” as used herein, refers to a minimal amount or concentration of an agent, composition, compound and/or drug that, when administered alone or in combination, is sufficient to provide a therapeutic benefit in the treatment of the condition, or to delay or minimize one or more symptoms associated with the condition. The term “therapeutically effective amount” can encompass an amount that improves overall therapy, reduces or avoids symptoms or causes of the condition, or enhances the therapeutic efficacy of another therapeutic agent. The therapeutic amount need not result in a complete cure of the condition; partial inhibition or reduction of the malignancy being treated is encompassed by this term.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes except to the extent they are inconsistent with the disclosures herein.

### DESCRIPTION OF THE DRAWINGS

FIG. 1—shows that ADAR1 induces cell cycle transit. Cord blood (CB) CD34⁺ cells were transduced with ADAR1 or ORF lentivirus. After 5-days, the cells were collected for FACS cell cycle analysis. An expansion of cells in S phase and decrease in G₀ were observed.

FIG. 2—shows that ADAR1 knock-down increases quiescence. CD34⁺ cells from CML BC patient were transduced with lentivirus expressing shADAR1 or shControl backbone for 3-days. FACS analysis indicates both the primary and serial engrafted samples have an increased G₀ population.

FIG. 3—(A) Plasmid map of fluorescent A-to-I editing reporter in pCDH lentivirus backbone; (B) Validation of A-to-I editing reporter in K562 using luciferase signal as readout. (C) Inactive ADAR1 mutant plasmid map. The RNA editing site of ADAR1 is mutated from A₅₂₉₃→C₅₂₉₃, which leads to glutamic acid (E) to alanine (A).

FIG. 4—shows that overexpression of ADAR1 in human stem cells leads to expansion in stem cell population in vitro (A) and (B) and in vivo (C).

FIG. 5—(A) shows the cloning scheme for generation of GLI2 lenti-viral constructs using pCDH-EF1-T2A-copGFP; (B) shows fold expression of GLI2 in SKNO-1 cells transduced with GLI2 lenti-viral construct; (C) shows Western blot analysis of cell extracts containing GLI2 lenti-viral constructs.

FIG. 6—shows generation of bi-cistronic FUCCI lenti-viral constructs using pCDH-EF1-T2A-copGFP. (A) FUCCI plasmids were used as templates to PCR subclone both Venus_hGeminin and mCherry_Cdt1 into pCDH-EF1-T2A lentiviral vector. (B) depiction showing the different fluorescent expression patterns of mCherry and mVenus in the cell cycle-with mCherry expressed primarily in G1 and mVenus in s and G2. (C) Fluorescence of the mCherry and mVenus FUCCI construct in cells.

FIG. 7—shows that ADAR1 drives leukemic progression by downregulating microRNA that target stem cell regulatory gene products.

FIG. 8—shows that microRNA are downregulated in CD34⁺ positive cells during CML progression from chronic phase to blast crisis.

FIG. 9—shows that lentiviral overexpression of ADAR1 leads to statistically significant downregulation of miRNA in primary CD34⁺ CML CP cells.

FIG. 10—lentiviral overexpression of ADAR1 in cord blood leads to significant downregulation of 16 microRNA.

FIG. 11—blast crisis CD34⁺ cells show downregulation of microRNA in common with cord blood cells overexpressing ADAR1. (A) downregulation of microRNA by overexpression of ADAR1 in cord blood; (B) comparison of microRNA expression in chronic phase versus blast crises phase showing downregulation of microRNA is similar to cord blood overexpressing ADAR1; (C) shows the ratio of p150 to p110 mRNA level in cord blood cells, CML chronic phase cells and CML blast crisis phase cells.

FIG. 12—shows that ADAR1 downregulation of LET7 family member increases self-renewal, and lenti-viral overexpression of LET7A induces a reduction in colony formation and replating. (A) shows overexpression of LET7A reduces colony number (B) shows percentage of colony (GM, G, BFU-E, MIX, and M) in cells transduce with a vector control or vector overexpressing LET7A. (C) shows number of replating colonies in cells with vector control or vector overexpressing LET7A.

FIG. 13—(A) shows relative mRNA expression of K562 cells WT, K562 lenti-ORF, or Lenti-ADAR1. (B) shows relative levels of ADAR1 isoforms (p150, p110), ADAR2 and RNA editing target gene MDM2 in wild-type (wt) K562, undifferentiated hESC (hues16 undiff), 293 cells and mouse bone marrow stromal cells (SL/M2).

FIG. 14—shows the selection of CSC-specific RNA editing biomarkers used for RESSq-PCR assay primer design.

FIG. 15—shows RNA editing site-specific qPCR (RESSqPCR) primer design strategy.

FIG. 16—shows RESSq-PCR primer sets. RNA editing site specific primer sets (non-genomic sequences) and control (positive) gene specific primers. Sites with primers listed as “n/a” failed the design parameters using the ARMS-based primer design.

FIG. 17—shows data for the validation of primer specificity of primers from FIG. 16 and Table 1 in cDNA and gDNA at RNA editing sites in MDM2, APOBEC3D, Gli1, GSK3b, and AZIN1 All primer sets generated single bands in cDNA by gel analysis.

FIG. 18—data showing RESSq-PCR detects robust RNA editing at ADAR target sites in K562-ADAR1 cDNA.

FIG. 19—shows validation by direct sequencing (ABOBEC3D-1). RESSq-PCR detects RNA editing at highly-edited sites in K562-ADAR1 RNA at similar ratios to direct sequencing.

FIG. 20—Gli1 direct Sequencing demonstrating a 10.81 fold increase by RESSq-PCR and 12.75 fold increase by direct sequencing (peak height ratio)

FIG. 21—shows inhibition of RNA editing in cord blood, ABM, and blast crisis cells with 8-azaadenosine. (A) 8-azaadenosine treatment results in reduced total ADAR1 levels by qRT-PCR, and (B) decreased RNA editing in APOBEC3D (Sites 1 & 2) by direct sequencing.

FIG. 22—shows let7 regulation by BCR-ABL and ADAR editing of miRs is niche dependent. Lentiviral over-expression of BCR-ABL in CD34+ CB n=3 do not affect expression of members of let7 family

FIG. 23—shows that all members of let7 family were downregulated in CB+BCR-ABL on stroma, while BCR-ABL effect was not consistent among the different members of let7 family in CB+ BCR-ABL without stroma. BCR-ABL overexpression affected CDKN1a expression when not on SLM2, while it affected CDKN 2A when in co-culture with SLM2

FIG. 24—shows let7 is regulated by JAK2 overexpression. (A) Let7-d was significantly downregulated after JAK2 overexpression, while V617F induced the upregulation of both let7e and let7f compared to JAK2 WT; (B) Fold change vs PCDH-JAK2 significantly downregulated 4 members of let-7 family and miR-155. Notably, no significant differences were observed between V617F transduced cells and PCDH; (C) Fold change vs lenti-JAK2 V617F mutation induced the upregulation of 3 members of let7 family.

FIG. 25 shows 8-azaadenosine derivatives.

FIG. 26—RNA editing inhibition with 8-azaadenosine in K562 cells stably expressing human ADAR1 p150. (A) qRT-PCR analysis of ADAR1 expression levels in K562 cells stably transduced with backbone control lentivirus, ADAR1 wild-type vector or ADAR1 mutant (catalytically deficient) vector. Cells were grown in the presence (+) of absence (−) of SLM2 humanized bone marrow stromal cells. (B, C) RNA editing analysis of 8-azaadenosine-treated (10-100 nM) cells (ADAR1 wt, B or ADAR1 mutant, C) using RESSq-PCR for a leukemia stem cell specific site in APOBEC3D.

FIG. 27—A) Correlation analysis between ADAR1 expression level and RNA editing of APOBEC3D by RESSqPCR in 293 cells transduced with ADAR1 WT and Mutant. Graph despicts best-fit line by Pearson correlation analysis. B) RESSqPCR analysis of APOBEC3D in normal CD34+ cord blood cells transduced with lenti-ADAR1 wild-type, lenti-ADAR1 mutant (editing inactive ADAR1) or backbone vector, and treated with 8-Aza for 22 hrs (n=3). C) mRNA expression levels of ADAR1 in normal CD34+ cord blood cells transduced with lenti-ADAR1 WT, lenti-ADAR1 mutant or backbone vector, after 22 hrs of treatment with 8-Aza (25 nM) (n=3).

### DETAILED DESCRIPTION OF THE EMBODIMENTS

Disclosed herein are compositions and methods useful for studying the cell cycle, RNA-editing enzymes, monitoring of disease progression, and pharmacological screening based on RNA editing detection. Also disclosed herein are research tools and methods useful to study stem cells and diseases associated with stem cells, including cancer stem cells. Specifically these tools involve ADAR1 constructs, GLI2 reporters, and cell-cycle fluorescent indicators.

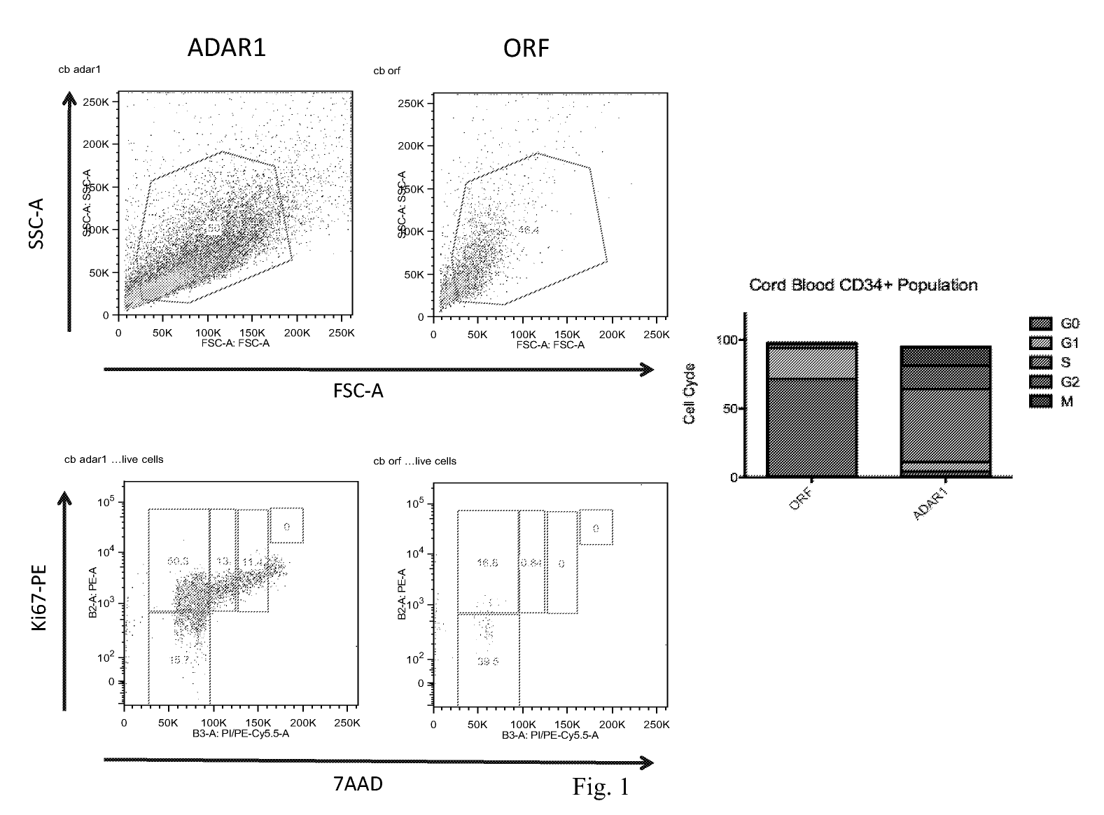

Data disclosed herein indicates that ADAR1 accelerates G0 to G1 phase transition in normal hematopoietic stem cells (cord blood CD34⁺ population), coupled with increased cell size and elevated expression of Ki67. The expanded population maintains stemness without any significant increase in differentiation. ADAR1 shows a preference of localization to the cell nucleus, suggesting the A-to-I editing events happen in the nucleus. qRT-PCR microarray of cell-cycle genes indicates that p21 expression level is reduced by >70% when ADAR1 is overexpressed. Therefore, ADAR1 is a useful tool for in vitro expansion of normal hematopoietic stem cells (FIG. 1). The data in FIG. 1 was obtained by transducing cord blood CD34⁺ cells with ADAR1 or ORF lentivirus. After 5-days, the cells were collected for FACS cell cycle analysis.

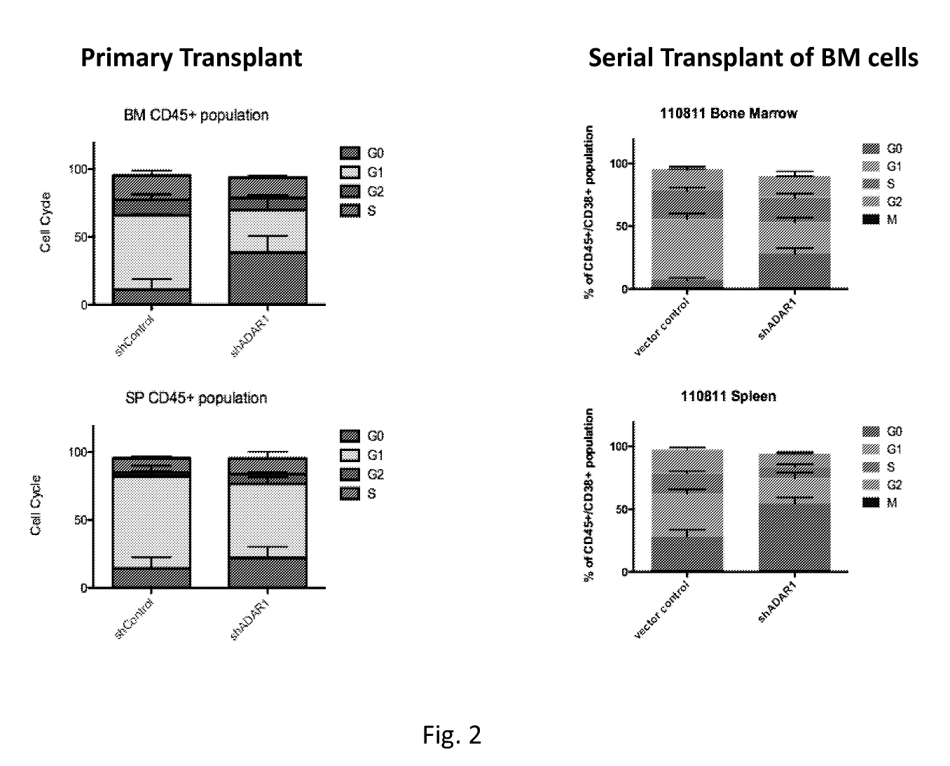

Moreover, shRNA knockdown of ADAR1 in CML BC sample shows a reduction of engraftment in bone marrow and spleen, and an enrichment of G0 population in the remaining cells. A decrease of self-renewal capacity as demonstrated by serial engraftment suggests the residual LSC failed to propagate (FIG. 2). The data in FIG. 2 was generated as follows: CD34⁺ cells from CML BC patient were transduced with lentivirus expressing shADAR1 or shControl backbone for 3-days. The cells were then transplanted into immunocompromised mice. After 20 weeks, the mice were sacrificed and the bone marrow (BM) and spleen (SP) were collected. CD34⁺ cells from bone marrow were serial transplanted to examine the self-renewal capacity. FACS analysis indicated both the primary and serial engrafted samples have an increased G0 population.

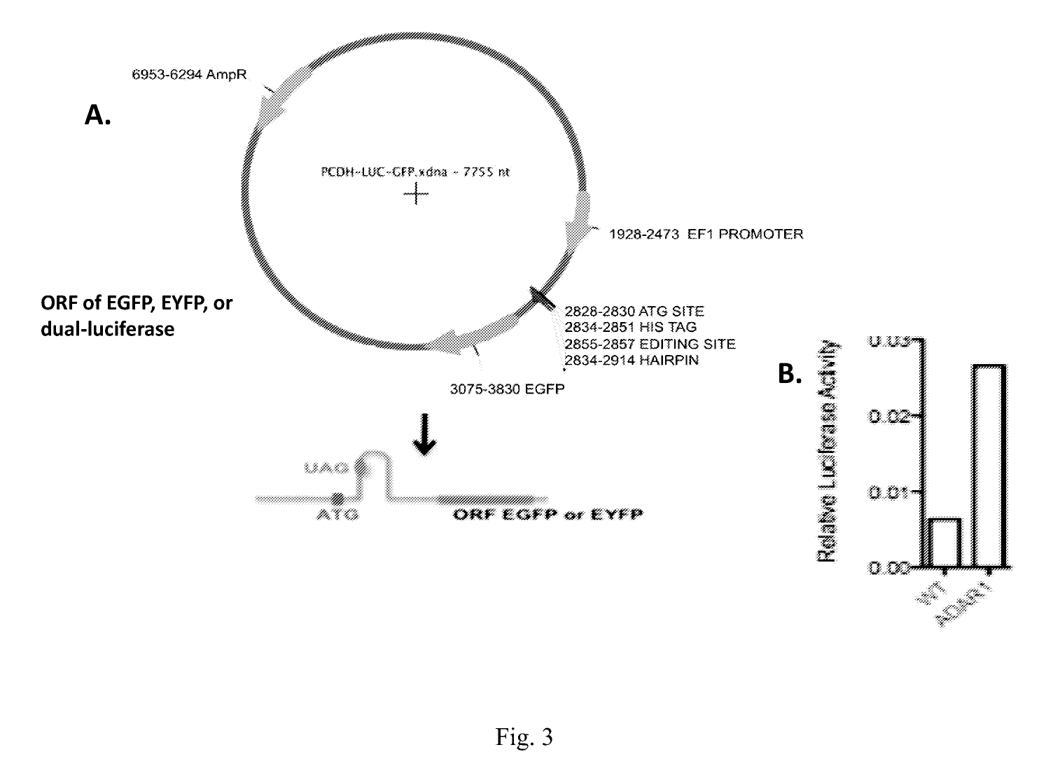

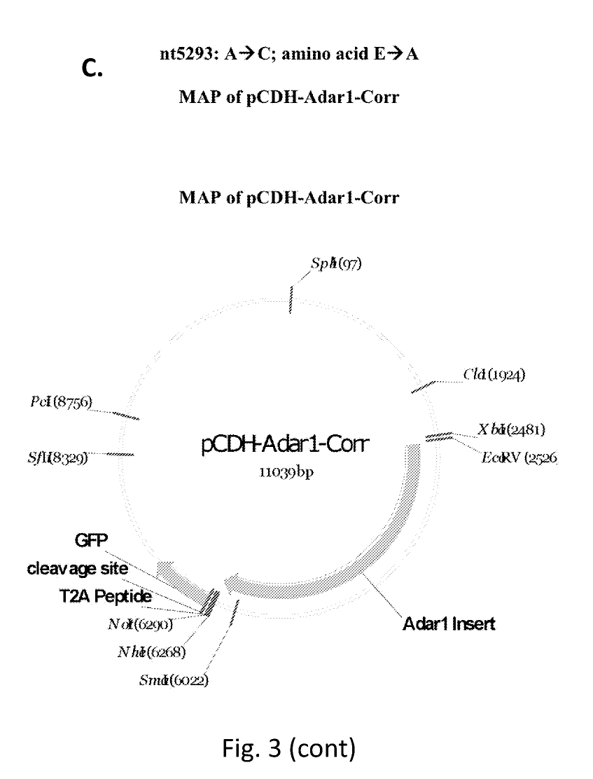

Also disclosed herein are vectors such as lentiviral vectors that overexpress a mutant version of ADAR1 protein. The point mutation locates in the active RNA-editing sites, and when mutated, the ADAR1 protein will retain dsDNA and dsRNA binding capacity, but lacks the ability to convert A-to-I. These constructs are useful for understanding the functionality of ADAR1 (FIG. 3)

In another embodiment, disclosed herein is a human-specific quantitative fluorescence dsRNA lentiviral reporter that can accurately measure aberrant A-to-I editing activity using luciferase activity or EGFP/YGFP as readout (see reference, Gommans et al, 2010). This reporter was cloned into a pCDH lentiviral plasmids (FIG. 3). The reporter contains a stop codon (TAG) in a hairpin structure as part of the promoter sequence. When RNA editing occurs, the stop codon is removed (TGG) and luciferase or fluorescent signal will serve as a readout of RNA editing level. In an aspect of this embodiment, an opposite orientated Alu-sequence is introduced into the hairpin structure to detect RNA editing in non-coding regions. The reporter's efficiency was confirmed in K562 leukemia cell line that stably overexpresses ADAR1 (approximate ˜10 fold by western blot). The results indicate a 5-fold increase in luciferase signal compared to K562 cells transduced with backbone lentivirus (FIG. 3B). In an aspect of this embodiment, the dsRNA reporter hairpin is introduced into a lentiviral backbone using luciferase activity or EGFP/EYFP as readout so that efficient transduction can be produced in quiescent myelodysplastic syndrome (MDS) and AML LSCs.

Such lentiviral A-to-I editing reporters can be used to measure and trace the A-to-I RNA editing changes in LCS and other CSC with in vitro stromal co-culture system and in vivo xenograft mouse models, and correlate these changes with exposure to agents such as toxins and other chemicals, including effects on therapeutic outcome.

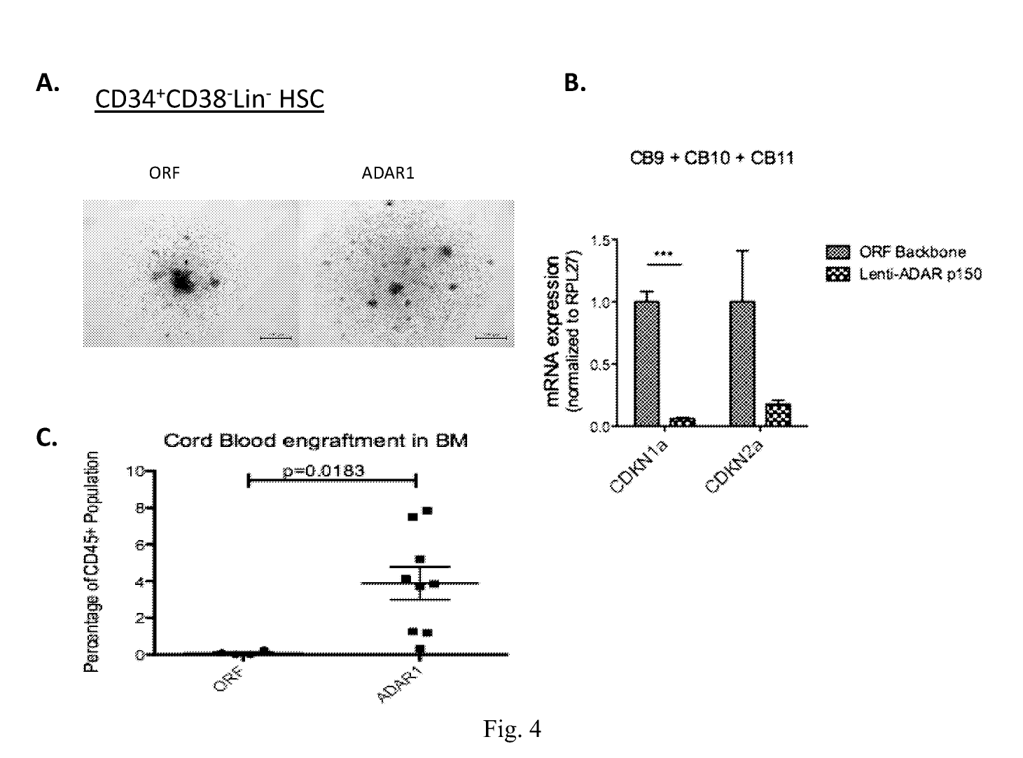

We have also observed an expansion cord blood cells ex vivo. This could serve as a new blood supply for patients who have had theirs destroyed by chemotherapy or radiation to treat leukemia, lymphoma and other blood-related diseases (FIG. 4). It has been difficult to successfully expand normal blood stem cells in vitro since the cells are likely to differentiate into mature blood cells. Our finding of ADAR1 indicates it is able to expand hematopoietic stem cells by 10-20 fold without significant increase in differentiation. It provides a safe and efficient method to produce large quantity of blood stem cell for bone marrow transplant and other medical needs, such for subjects who have had their blood cells destroyed by chemotherapy or radiation to treat, without limitation, leukemia, lymphoma and other blood-related diseases.

In one embodiment, the procedure can start with collection of cord blood cells from a donor or donors which is then transduced with lentivirus that overexpresses ADARI RNA editing enzyme. After about 3-5 days, the cells are collected and transplanted back into patients who received therapy to enable a healthy blood system to reestablish quickly. In some aspects the transplanted cells are CD34⁺. Successful expansion can be determined, without limitation, by measuring the elevation in CD45⁺ (FIG. 4). This method enables quick transplantation of donor blood cells immediately after patients receive chemotherapy or radiation. The increase in number of healthy donor blood also helps to provide a faster and better recovery. Since the culture condition is very simple and the turn-over-time is short, this method provides a more efficient alternative to existing technologies.

The data in FIG. 4 was obtained as follows: FACS-purified CD34⁺ CD38⁻Lin⁻ HSC (n=3) were transduced with ADAR1 or ORF backbone. The pictures were taken 3-days post transduction (FIG. 4A), when the cells were collected for qRT-PCR analysis. Genes involved in cell cycle arrest (CDKN1a and CDKN2a) were both down-regulated by ADAR1 (FIG. 4B). Cord blood CD34⁺ cells were transduced with ADAR1 or ORF and transplanted into immunodeficient mice. After 12 weeks, bone marrow were collected and analyzed by FACS (FIG. 4C).

### Cell-Cycle Reporters

Currently, there are few methods to visualize cell cycle progression in LSC. Human LSC are characterized as quiescent self-renewing cells that drive leukemic transformation of myeloproliferative neoplasms and myelodysplastic syndromes. Additionally, current methods require sequential transduction and clonal selection. These methods and conditions are not feasible to study primary patient samples and LSC, which are generally limited in sample size. To alleviate this challenge, we generated a lentiviral biscistronic vector encoding FUCCI (fluorescent ubiquitination-based cell-cycle indicator) probes. The development of this new reporter offers a new and more effective method to track cell cycle progression in limited mammalian cell populations, including cell cycle changes in CSC. These constructs can identify changes in the cell cycle leading to new strategies to combat chemoresistance, and for screening new candidate therapeutics.

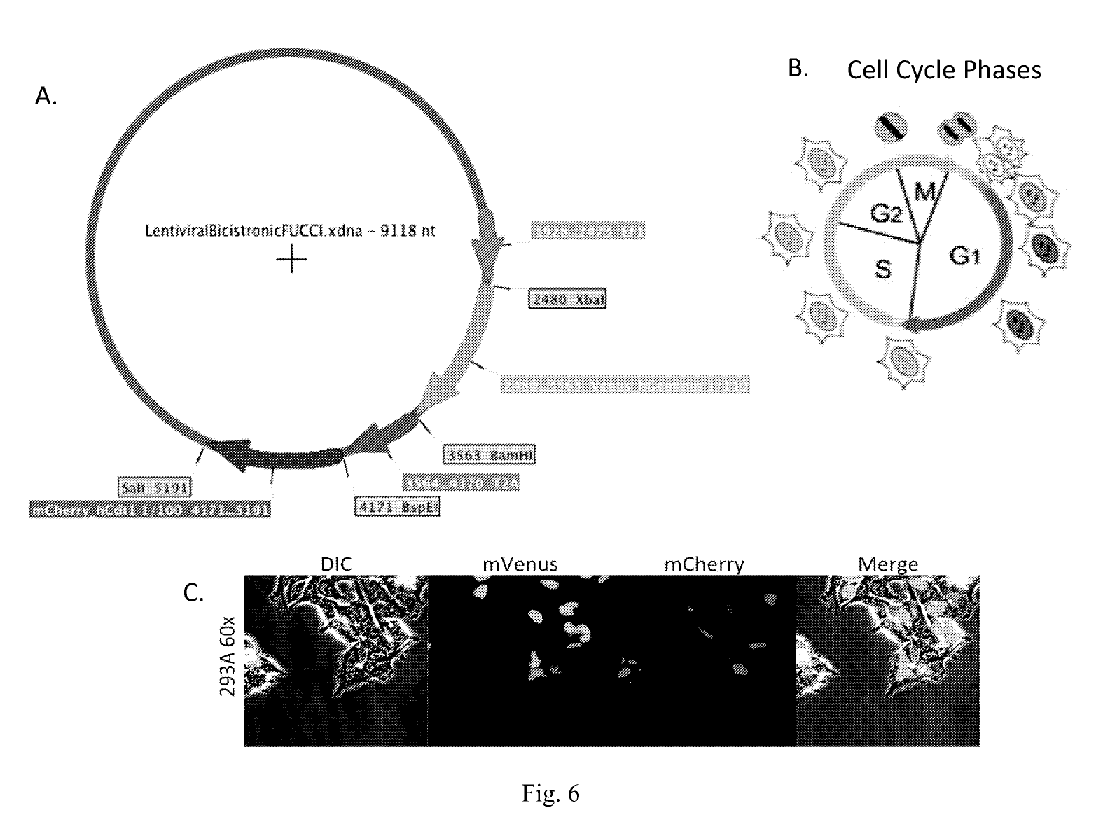

Bi-cistronic FUCCI reporter was generated by subcloning mCherry-hCdt1 into BspE1/SalI digested pcDH-EF1-T2A-copGFP and ligated in frame. Venus-hGeminin was subcloned into XbaI/BamHI digested pcDH-EF1-T2A-mCherry-hCdt1 and ligating in frame (FIG. 6).

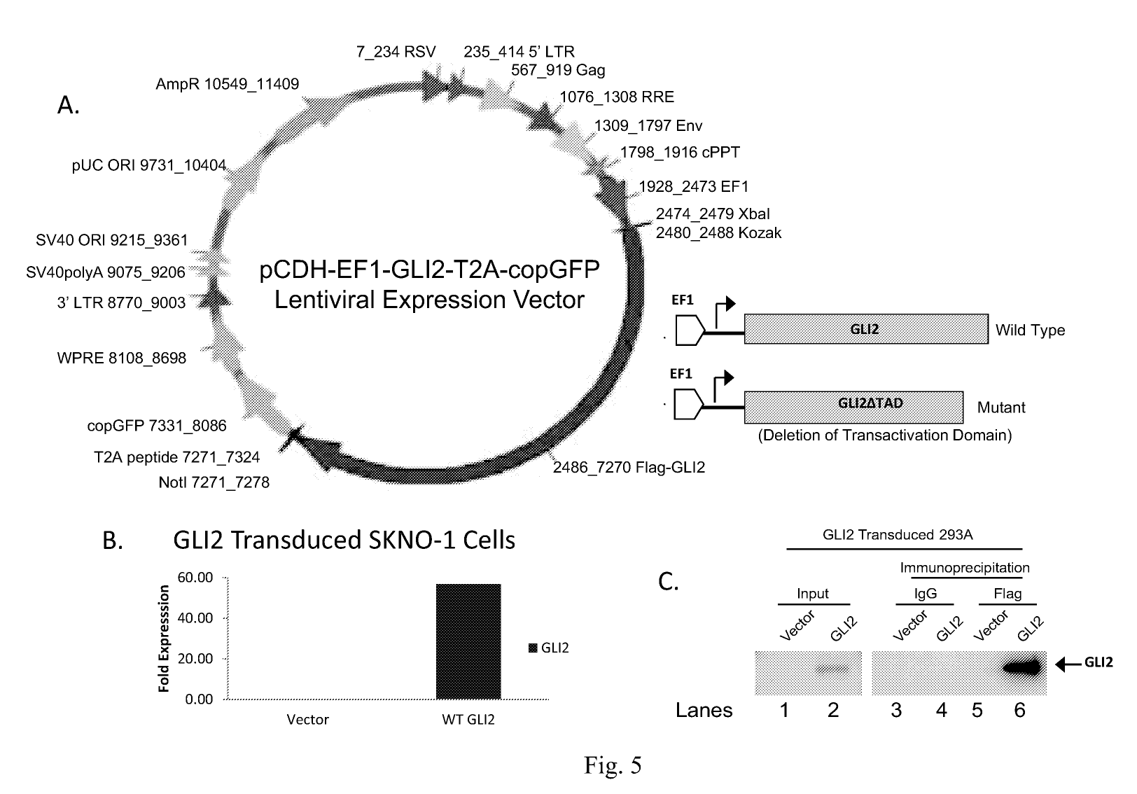

GLI2 Wild Type and mutant GLI2 Vectors: Wild type and mutant versions of GLI2 were generated by either subcloning full length human GLI2 or transcriptionally inactive GLI2 into XbaI/NotI digested pcDH-EF1-T2A-copGFP and ligated in frame with the FLAG epitope. Mutant GLI2 was generated by deletion of transcriptional activation domain of GLI2 denoted as pcDH-EF1-ΔTAD GLI2-T2A-copGFP (FIG. 5). FIG. 5B shows the fold expression of GLI2 in GLI2 transduced SKNO-1 cells using the GLI2 vector. FIG. 5C show Western blots of cells transduced with the GLI2 vector.

### Micro RNA:

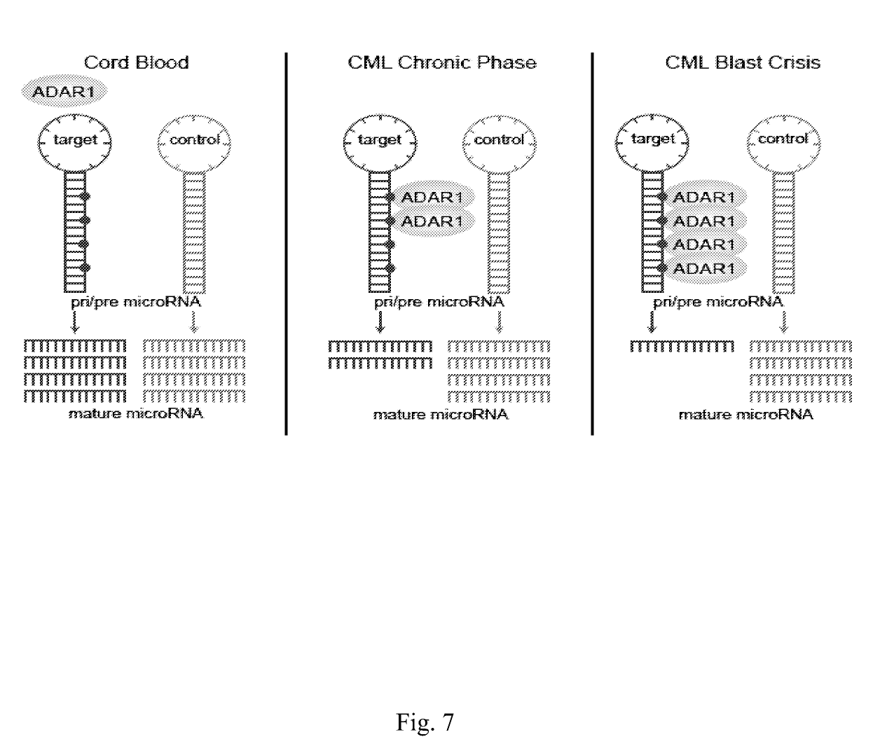

While miRNA alterations have been associated with CML in the context of bulk tumor, no published studies have focused on CML LSC population that behaves remarkably different from bulk tumor and is responsible for disease progression and relapse. Disclosed herein is the finding that in Chronic Myeloid Leukemia (CML) progression is driven by ADAR1-dependent regulation of microRNA. Without wishing to be bound by any particular theory, it is postulated that in blast crisis, when ADAR1 p150 is upregulated, ADAR1 affects microRNA regulation by editing of pri-microRNA and pre-microRNA (FIG. 7). This discovery is unique since it allows one to predict leukemia progression by identifying edited miRNA generated by malignant-specific A-to-I RNA editase ADAR1. This will also allow one to use edited miRNA signature, instead of a general miRNA profile as disease biomarkers. Moreover, the identification of disease progression markers can help identify and develop new targeted therapies. In an aspect of this embodiment, this discovery allows for the development of a detection platform of edited microRNA in LSC population from primary patients and to validate their role as diagnostic and prognostic biomarkers with primary leukemia patient at different disease stages and following different treatments.

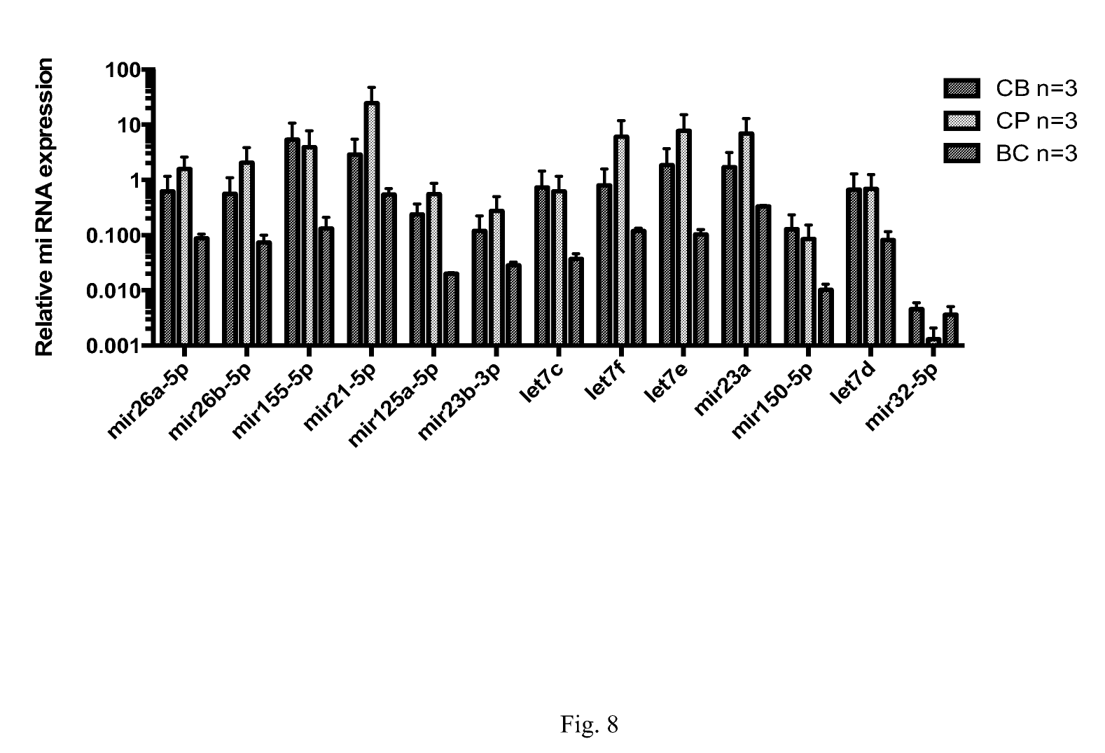

Also, disclosed herein is finding that blast crisis CD34⁺ cells show downregulation of 13 microRNA compared to CD34⁺ Chronic Phase. This can be related to overexpression of ADAR1 in Blast Crisis. ADAR1, by editing microRNA precursors negatively affect complete maturation of microRNA mediated by RNAse Drosha and DICER, thus leading to degradation of the edited product and a consequent downregulation of the edited microRNA (FIG. 8). The data in FIG. 8 was generated as follows: Primary CML patient samples and normal blood were CD34 selected for RNA extraction. cDNA was prepared in a reverse-transcription reaction using miScript RTII kit (QIAGEN) and used as a template to profile the expression of the 84 most abundantly expressed and best characterized miRNAs by using miScript miRNA PCR Array (QIAGEN), which contains miRNA specific miScript primer assays. qRT-PCR was performed with SYBR Green Kit (QIAGEN). qRT-PCR for the validation of array results was performed by using miRNA specific primer assays and SYBR Green Kit (QIAGEN). Normalization was performed by using miSCRIPT primer control (RNU6-2).

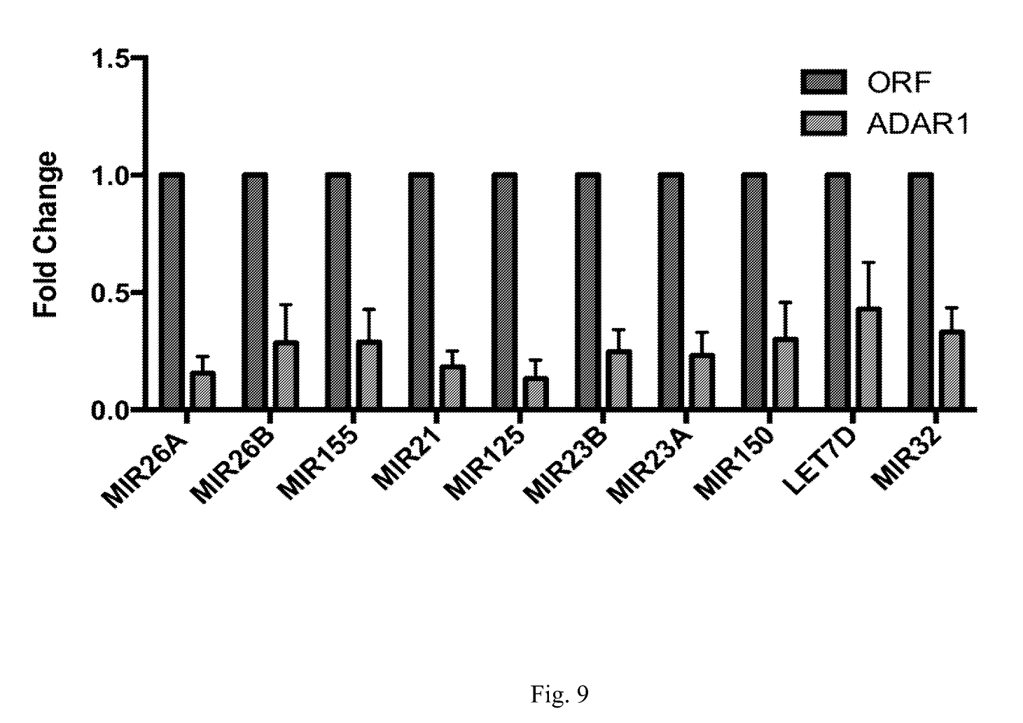

Another embodiment disclosed herein is the finding that Lenti-viral overexpression of ADAR1 in chronic phase primary samples leads to a statistically significant downregulation of 10 microRNA, thus, showing a microRNA expression profile more similar to that seen in blast crisis (FIG. 9). The data generated in FIG. 9 was obtained as follows: CP CD34⁺ cells were transduced with lentiviral vectors overexpressing human ADAR1 or GFP-expressing lentiviral backbone. Cells were collected for RNA extraction and MiRNA expression was evaluated by MiScript qPCR array, and differentially expressed miRNAs was validated by qRT-PCR with specific primers.

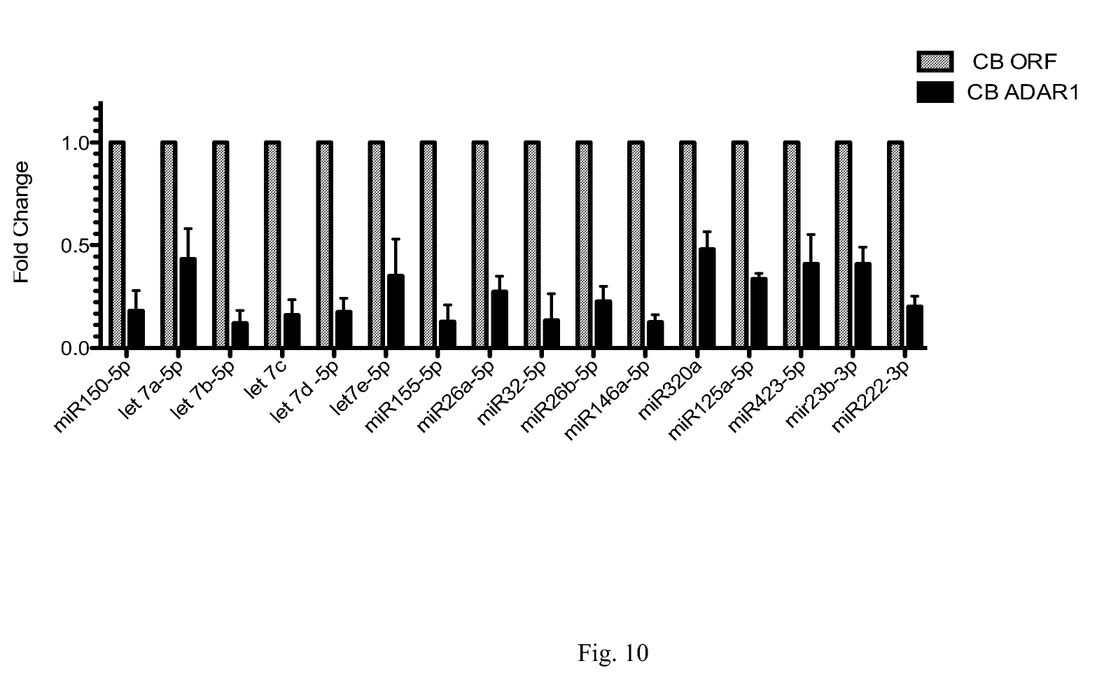

Lenti-viral overexpression of ADAR1 in CD34⁺ Cord blood cells leads to the significant downregulation of 16 microRNA (FIG. 10). The data shown in FIG. 10 was generated as follows CP CD34⁺ cells were transduced with lentiviral vectors overexpressing human ADAR1 or GFP-expressing lentiviral backbone. Cells were collected for RNA extraction and MiRNA expression was evaluated by MiScript qPCR array, and differentially expressed miRNAs was validated by qRT-PCR with specific primers.

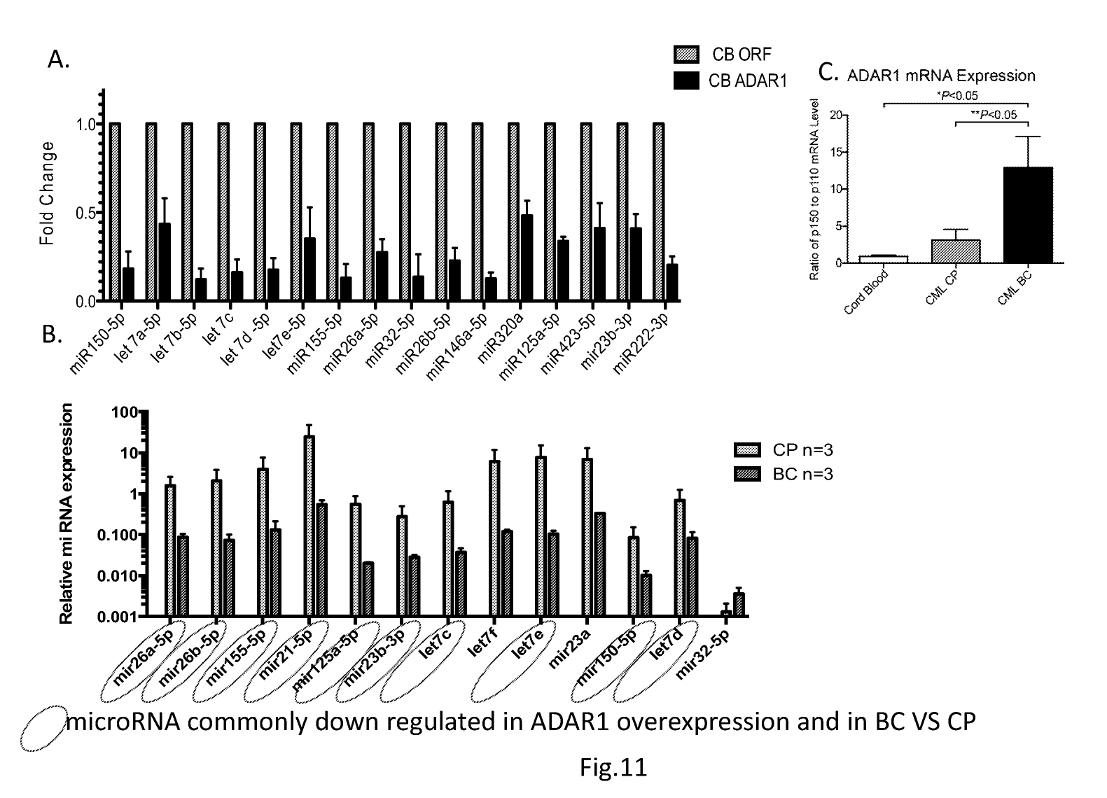

In Blast Crisis, where ADAR1 is upregulated and induces malignant progenitor reprogramming, 10 out the 13 microRNA that are downregulated in comparison with chronic phase, are in common with the downregulated microRNA in ADAR1 overexpressing Cord blood (FIG. 11).

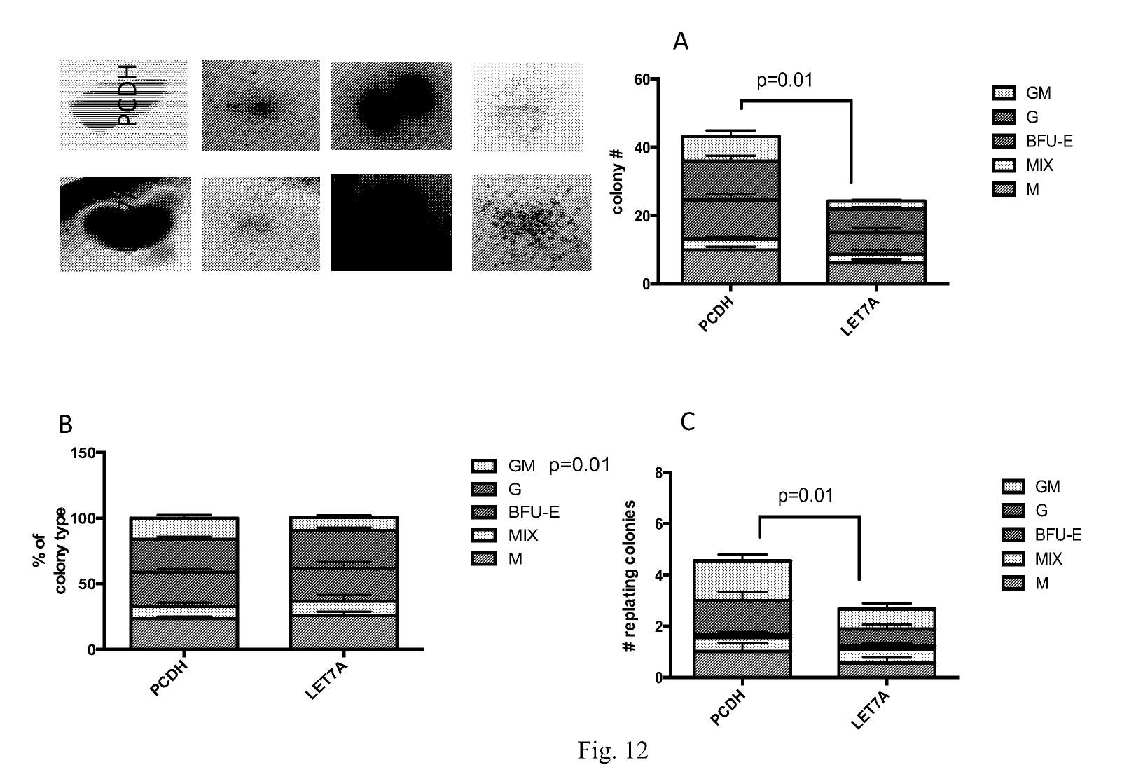

Since in both ADAR1 transduced cord blood and blast crisis, we observed a downregulation of the family of reprogramming microRNA let7, we overexpressed let7a in cord blood and evaluated the expression of the target LIN28, as well as its effect on self-renewal. Let7a overexpression leads to a statistical significant decrease of the efficiency of colony formation. This reduction is driven by the reduction in GM colonies. Moreover, overexpression of let7a decreases self-renewal as shown by the reduction of number of replating colonies. Further, Lin28 is statistically downregulated after let7-a overexpression. ADAR1, by downregulating let7 family increases self-renewal (FIG. 12). The data shown in FIG. 12 was obtained as follows: CD34⁺ cord blood n=3 cells were transduced with lentivirus overexpressing let-7a or GFP expressing lentiviral backbone. After 48 hours Hematopoietic progenitor assays was performed by plating cells into MethoCult Medium. After 2 weeks, total colony number FIG. 12A and change in individual colony types Fig. B between miRNA-overexpressing and the control conditions was analyzed. Individual colonies were replated into fresh Methocult medium and replating efficiency after 2 weeks of culture was evaluated as a measure of self-renewal capacity (n=2) FIG. 12C.

### RESSq-PCR and RNA Editing Inhibitors:

Whole transcriptome RNA sequencing (RNA-Seq) revealed increased adenosine to inosine (A-to-I) RNA editing during CML progression concentrated within primate specific Alu-containing transcripts. However, detection of RNA editing by RNA-Seq in rare cell populations can be technically challenging, costly and requires PCR validation by direct sequencing which cannot reliably detect low levels of RNA editing, or cloning which is labor-intensive and low-throughput. RNA Editing Site-Specific qPCR (RESSq-PCR) provides a rapid, clinically amenable method to detect aberrant primate-specific RNA editing at CSC-associated loci.

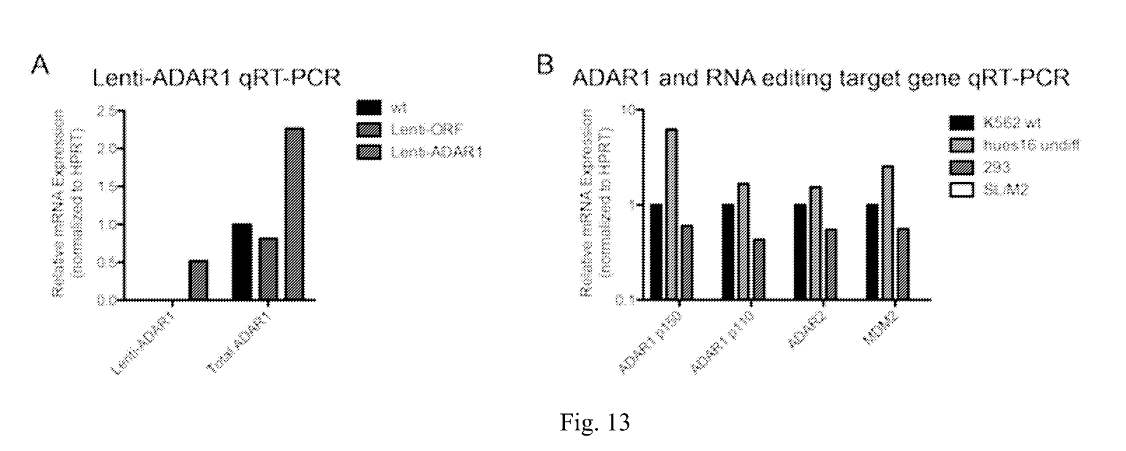

To develop a model system for development of the RESSq-PCR diagnostic assay, we established an in vitro model of stable ADAR1 expression in leukemia cells, wherein the BCR-ABL⁺ human leukemia cell line K562 was stably transduced with lentiviral human ADAR1-IRES-GFP (K562-ADAR1) or vector ORF (K562-ORF). Positively-transduced cells were identified on the basis of high GFP expression, FACS-purified to establish stable cell lines, and expanded under routine culture conditions. High levels of ADAR1 lentivirus and total human ADAR1 mRNA in K562-ADAR1 cells were confirmed by qRT-PCR using lentiviral-specific and human-specific primers, respectively (FIG. 13). Cell types with high levels of endogenous ADAR1 expression (undifferentiated human embryonic stem cells) were used as a positive control for comparison to enforced ADAR1 overexpression in K562 cells (FIG. 13). For the data shown in FIG. 13A, K562 cells were transduced with lentiviruses expressing human ADAR1-GFP (Lenti-ADAR1) or backbone-GFP (Lenti-ORF) control. Cells were FACS-purified for GFP-positive cells and expanded in culture. 106 cells were harvested and RNA was extracted and reverse transcribed using a cDNA supermix containing random hexamers and oligodT. ADAR1 lentivirus and total ADAR1 levels were measured by qPCR. In FIG. 13B relative levels of ADAR1 isoforms (p150, p110), ADAR2 and RNA editing target gene MDM2 were evaluated in wild-type (wt) K562, undifferentiated hESC (hues16 undiff), 293 cells and mouse bone marrow stromal cells (SL/M2).

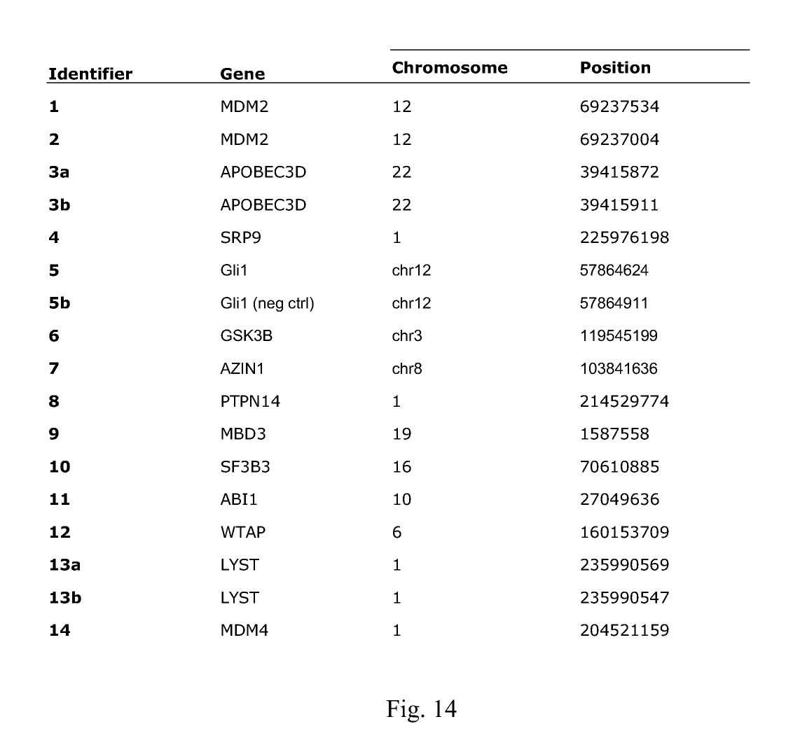

We previously identified 274 differentially edited sites in CML blast crisis versus chronic phase, and selected top candidates from this dataset to develop site-specific primers and validate RNA editing sites as potential biomarkers of disease progression in primary patient samples. Candidate sites for validation and qRT-PCR assay development were selected from our previous RNA-sequencing data on the basis of greatest average change in RNA editing, with a focus on selecting sites within functionally relevant target genes involved in stem cell survival and self-renewal pathways, along with 3 known RNA editing sites within stem cell regulatory genes and cancer-associated pathways (FIG. 14).

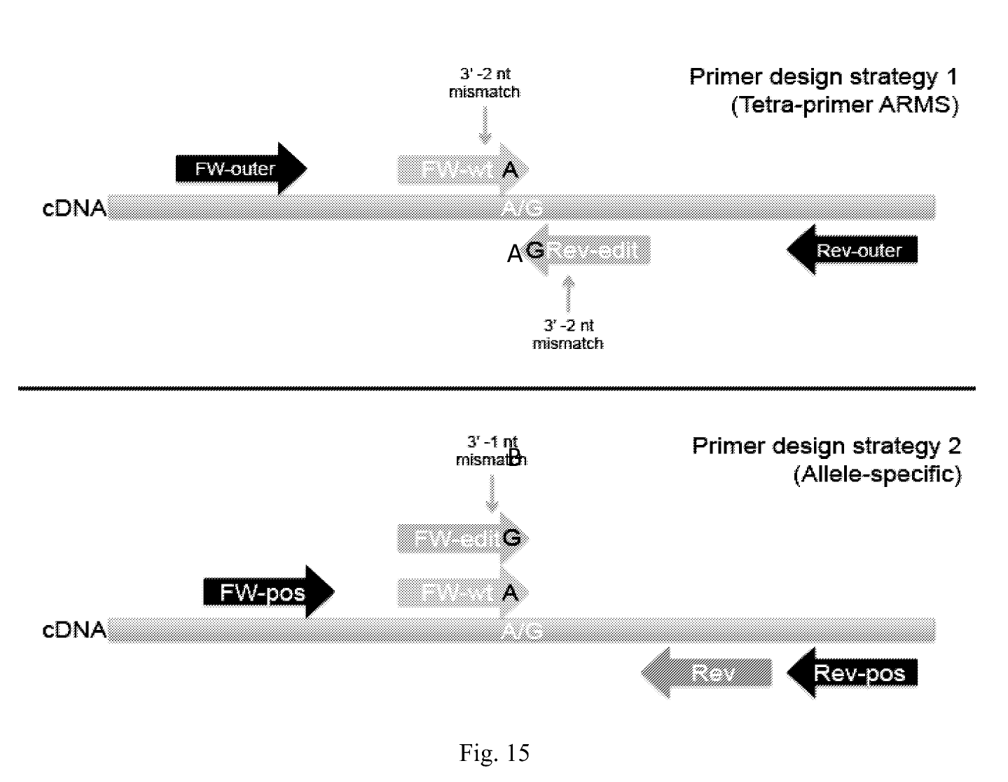

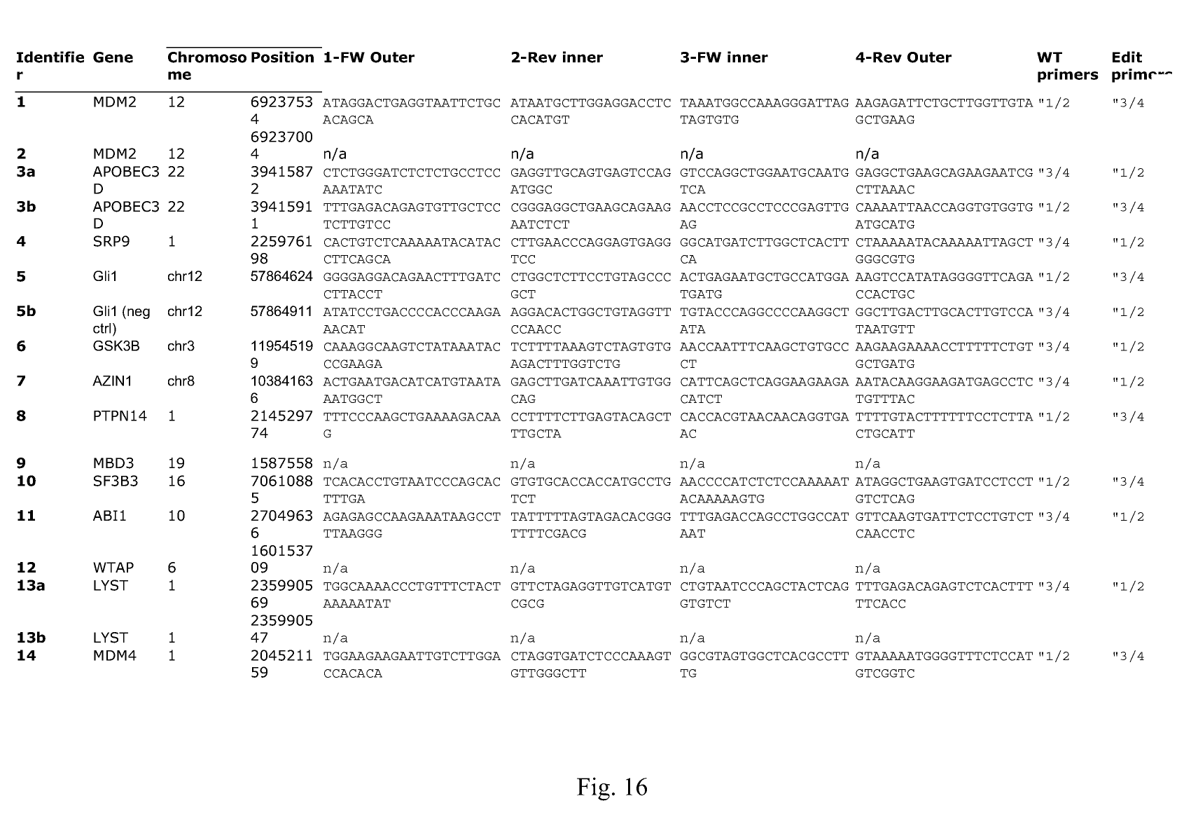

In order to implement rapid, inexpensive and clinically-amenable detection of RNA-edited transcripts that predict leukemic progression, we utilized a novel RNA editing site-specific quantitative RT-PCR (RESS-qPCR) primer design strategy. Since RNA-edited transcripts are predicted to differ at only one nucleotide position, developing highly specific primers for traditional qRT-PCR assays requires sensitive and selective primer design strategies. We have previously developed qRT-PCR primers that specifically recognize a gene product with one point mutation (JAK2 V617F11), and here we employed a similar strategy in designing RESSq-PCR primers (FIG. 15 and FIG. 16). For each ADAR target site, two sets of primers containing non-genomic sequences were designed: one pair detecting the wild-type transcript (an “A” base), and one pair detecting the edited transcript (a “G” base representing inosine substitution) (FIG. 16 and Table 1). An additional mismatch was incorporated upstream of the 3′ primer end to enhance allelic discrimination.

TABLE 1
RESSq-PCR primer sets

| Gene | 1-FW Outer | 2-Rev inner | 3-FW inner | 4-Rev Outer |
|---|---|---|---|---|
| MDM2 | ATAGGACTGAGGT | ATAATGCTTGGAG | TAAATGGCCAAAG | AAGAGATTCTGCT |
| | AATTCTGCACAGC | GACCTCCACATGT | GGATTAGTAGTGT | TGGTTGTAGCTGA |
| | A | SEQ ID NO: 2 | G | AG |
| | SEQ ID NO: 1 | | SEQ ID NO: 3 | SEQ ID NO: 4 |
| MDM2 | n/a | n/a | n/a | n/a |
| APOB | CTCTGGGATCTCT | GAGGTTGCAGTGA | GTCCAGGCTGGAA | GAGGCTGAAGCAG |
| EC3D | CTGCCTCCAAATA | GTCCAGATGGC | TGCAATGTCA | AAGAATCGCTTAA |
| | TC | SEQ ID NO: 6 | SEQ ID NO: 7 | AC |
| | SEQ ID NO: 5 | | | SEQ ID NO: 8 |
| APOB | TTTGAGACAGAGT | CGGGAGGCTGAAG | AACCTCCGCCTCC | CAAAATTAACCAG |
| EC3D | GTTGCTCCTCTTG | CAGAAGAATCTCT | CGAGTTGAG | GTGTGGTGATGCA |
| | TCC | SEQ ID NO: 10 | SEQ ID NO: 11 | TG |
| | SEQ ID NO: 9 | | | SEQ ID NO: 12 |
| SRP9 | CACTGTCTCAAAA | CTTGAACCCAGGA | GGCATGATCTTGG | CTAAAAATACAAA |
| | ATACATACCTTCA | GTGAGGTCC | CTCACTTCA | AATTAGCTGGGCG |
| | GCA | SEQ ID NO: 14 | SEQ ID NO: 15 | TG |
| | SEQ ID NO: 13 | | | SEQ ID NO: 16 |
| Gli1 | GGGGAGGACAGAA | CTGGCTCTTCCTG | ACTGAGAATGCTG | AAGTCCATATAGG |
| | CTTTGATCCTTAC | TAGCCCGCT | CCATGGATGATG | GGTTCAGACCACT |
| | CT | SEQ ID NO: 18 | SEQ ID NO: 19 | GC |
| | SEQ ID NO: 17 | | | SEQ ID NO: 20 |
| Gli1 | ATATCCTGACCCC | AGGACACTGGCTG | TGTACCCAGGCCC | GGCTTGACTTGCA |
| (neg | ACCCAAGAAACAT | TAGGTTCCAACC | CAAGGCTATA | CTTGTCCATAATG |
| ctrl) | SEQ ID NO: 21 | SEQ ID NO: 22 | SEQ ID NO: 23 | TT |
| | | | | SEQ ID NO: 24 |
| GSK3B | CAAAGGCAAGTCT | TCTTTTAAAGTCT | AACCAATTTCAAG | AAGAAGAAAACCT |
| | ATAAATACCCGAA | AGTGTGAGACTTT | CTGTGCCCT | TTTTCTGTGCTGA |
| | GA | GGTCTG | SEQ ID NO: 27 | TG |
| | SEQ ID NO: 25 | SEQ ID NO: 26 | | SEQ ID NO: 28 |
| AZIN1 | ACTGAATGACATC | GAGCTTGATCAAA | CATTCAGCTCAGG | AATACAAGGAAGA |
| | ATGTAATAAATGG | TTGTGGCAG | AAGAAGACATCT | TGAGCCTCTGTTT |
| | CT | SEQ ID NO: 30 | SEQ ID NO: 31 | AC |
| | SEQ ID NO: 29 | | | SEQ ID NO: 32 |
| PTPN14 | TTTCCCAAGCTGA | CCTTTTCTTGAGT | CACCACGTAACAA | TTTTGTACTTTTT |
| | AAAGACAAG | ACAGCTTTGCTA | CAGGTGAAC | TCCTCTTACTGCA |
| | SEQ ID NO: 33 | SEQ ID NO: 34 | SEQ ID NO: 35 | TT |
| | | | | SEQ ID NO: 36 |
| MBD3 | n/a | n/a | n/a | n/a |
| SF3B3 | TCACACCTGTAAT | GTGTGCACCACCA | AACCCCATCTCTC | ATAGGCTGAAGTG |
| | CCCAGCACTTTGA | TGCCTGTCT | CAAAAATACAAAA | ATCCTCCTGTCTC |
| | SEQ ID NO: 37 | SEQ ID NO: 38 | AGTG | AG |
| | | | SEQ ID NO: 39 | SEQ ID NO: 40 |
| ABI1 | AGAGAGCCAAGAA | TATTTTTAGTAGA | TTTGAGACCAGCC | GTTCAAGTGATTC |
| | ATAAGCCTTTAAG | CACGGGTTTTCGA | TGGCCATAAT | TCCTGTCTCAACC |
| | GG | CG | SEQ ID NO: 43 | TC |
| | SEQ ID NO: 41 | SEQ ID NO: 42 | | SEQ ID NO: 44 |
| WTAP | n/a | n/a | n/a | n/a |
| LYST | TGGCAAAACCCTG | GTTCTAGAGGTTG | CTGTAATCCCAGC | TTTGAGACAGAGT |
| | TTTCTACTAAAAA | TCATGTCGCG | TACTCAGGTGTCT | CTCACTTTTTCAC |
| | TAT | SEQ ID NO: 46 | SEQ ID NO: 47 | C |
| | SEQ ID NO: 45 | | | SEQ ID NO: 48 |
| LYST | n/a | n/a | n/a | n/a |
| MDM4 | TGGAAGAAGAATT | CTAGGTGATCTCC | GGCGTAGTGGCTC | GTAAAAATGGGGT |
| | GTCTTGGACCACA | CAAAGTGTTGGGC | ACGCCTTTG | TTCTCCATGTCGG |
| | CA | TT | SEQ ID NO: 51 | TC |
| | SEQ ID NO: 49 | SEQ ID NO: 50 | | SEQ ID NO: 52 |

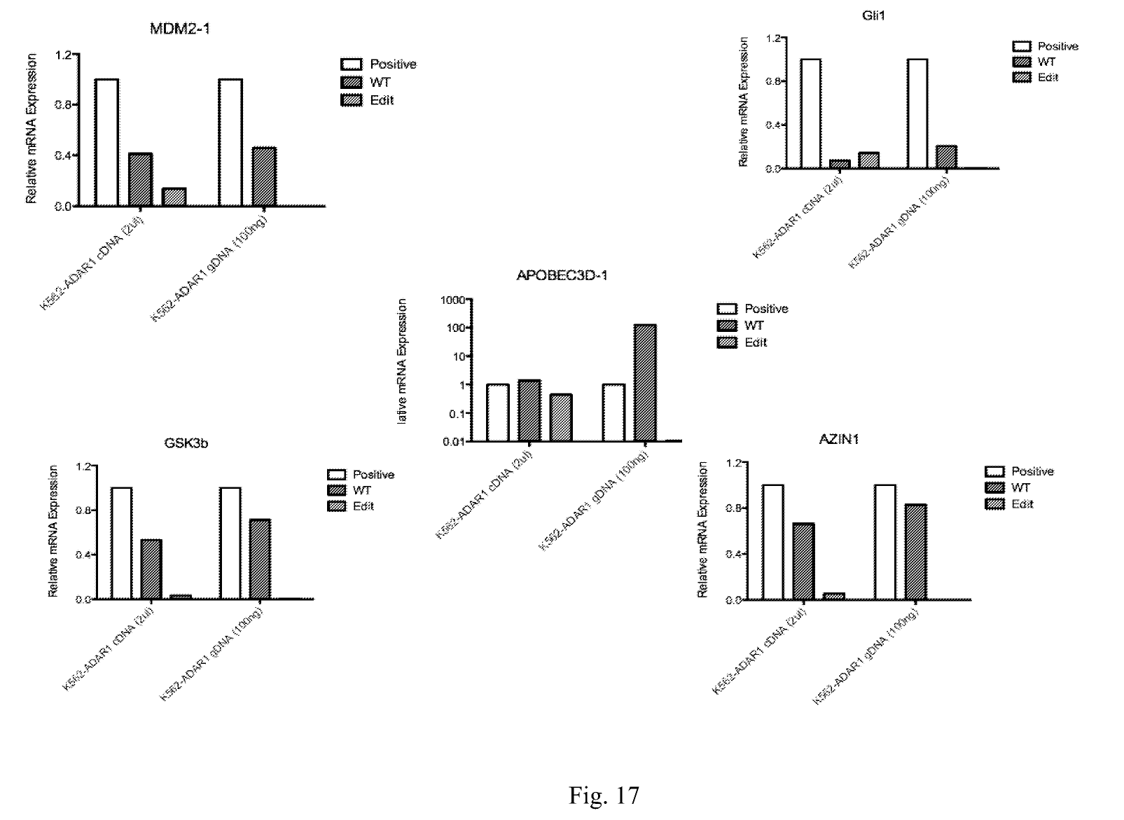

In FIG. 17, cDNA and genomic DNA (gDNA) from K562-ADAR1 cells were analyzed by RESSq-PCR with primers specific for wild-type (WT, A), edit (G) or positive (outer flanking) gene sequences at RNA editing sites in MDM2, APOBEC3D, Gli1, GSK3b, and AZIN1 Site-specific primers distinguished G(I) bases at RNA editing sites in cDNA and as predicted gave no signal in gDNA (FIG. 17).

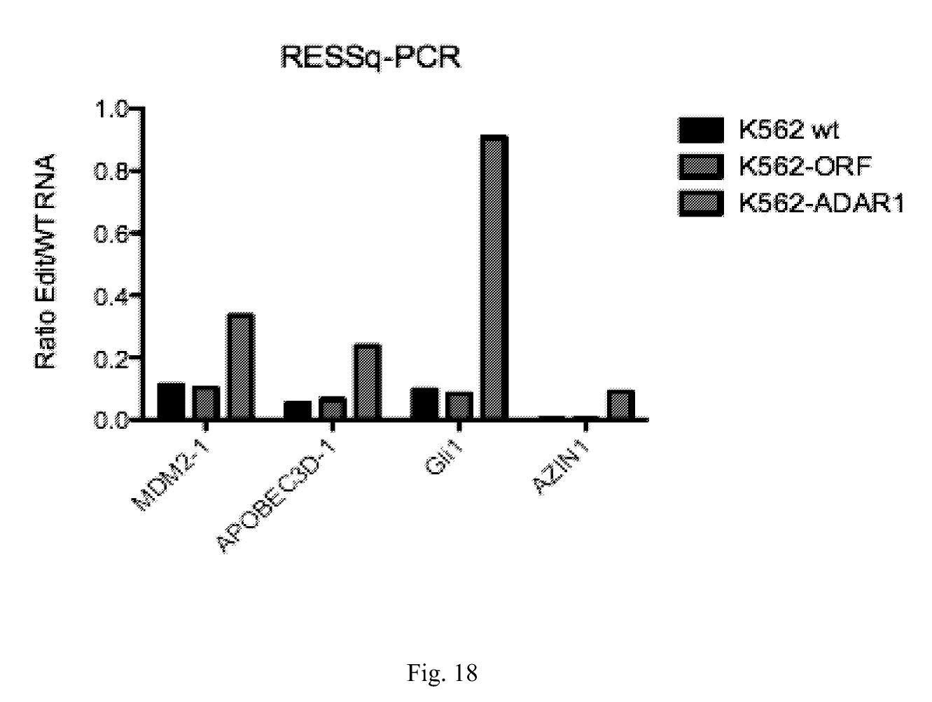

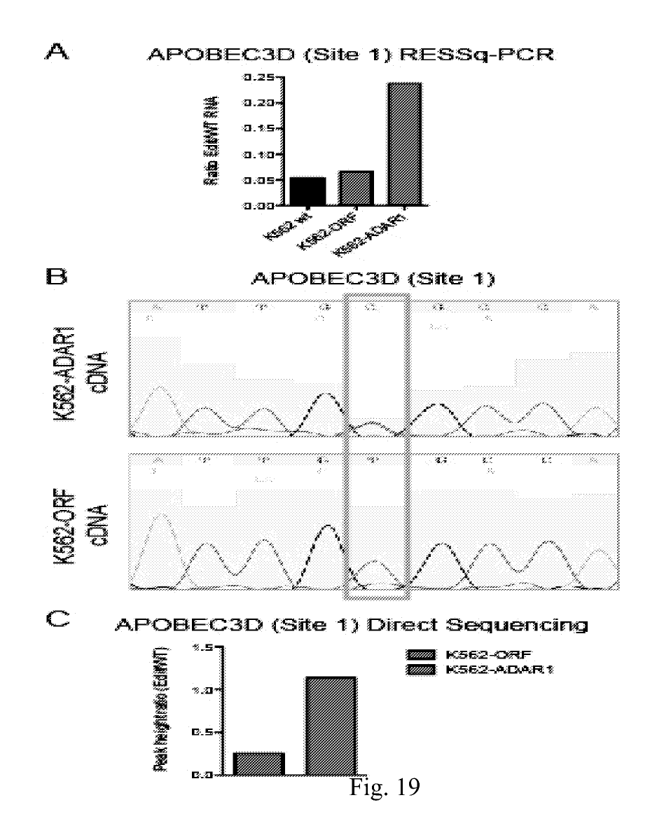

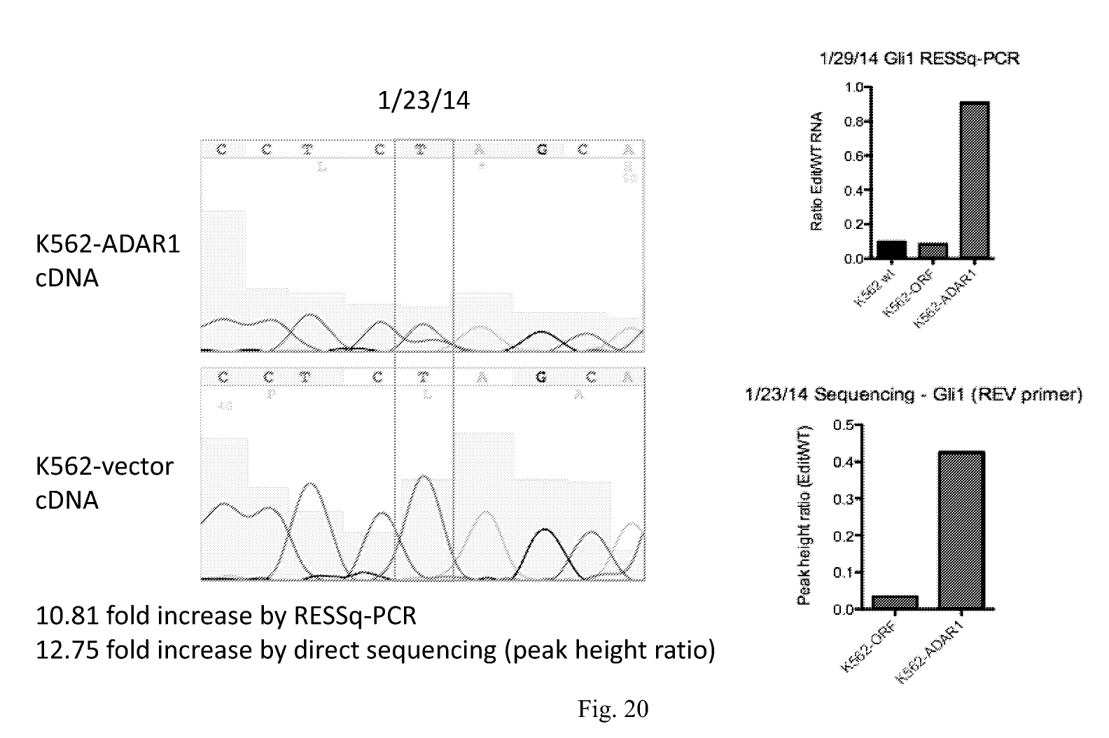

All primer sets generated single bands in cDNA by gel analysis. In independent experiments, RESSq-PCR accurately detected robust RNA editing in K562-ADAR1 cells (FIG. 18). Relative A-to-I RNA editing ratios were increased by 2 to 3 fold in ADAR1-expressing cells compared to vector controls (ORF) at sites in MDM2, APOBEC3D, Gli1 and AZIN1 transcripts (FIG. 18). Increased A-to-I changes in RNA editing sites were confirmed by targeted sequencing (FIGS. 19, 20).

### Small Molecule Inhibitors:

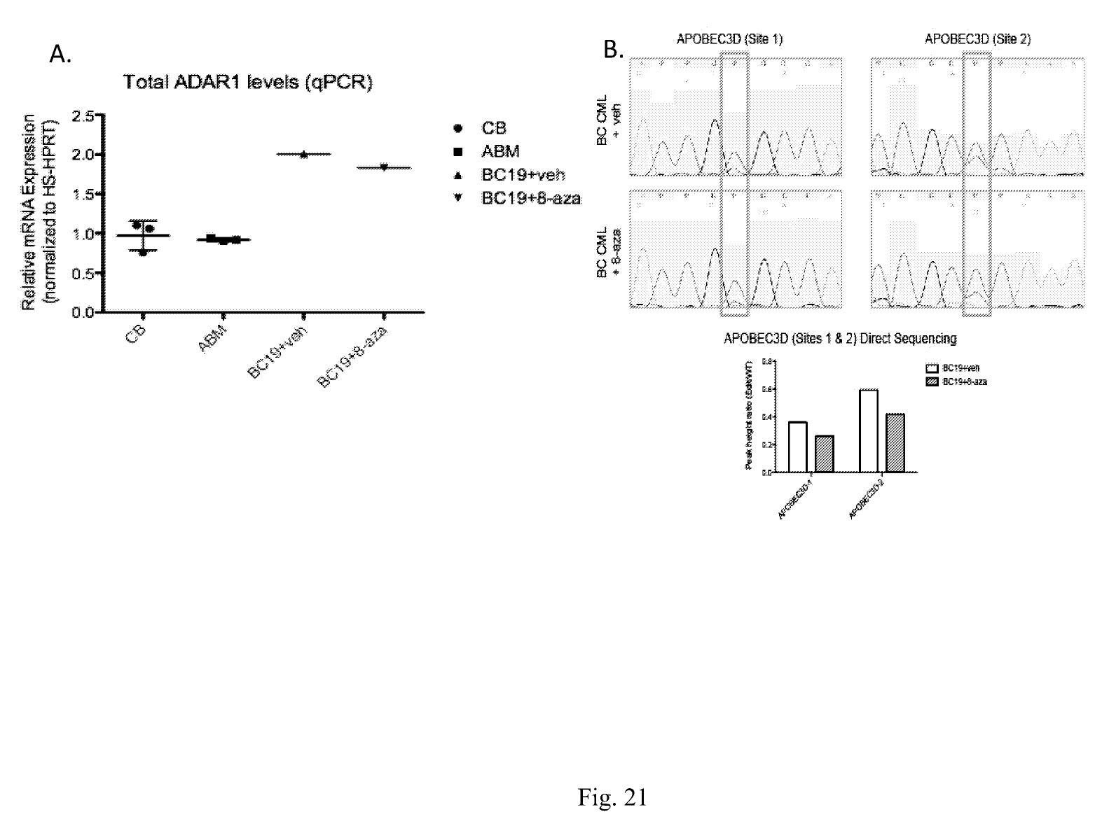

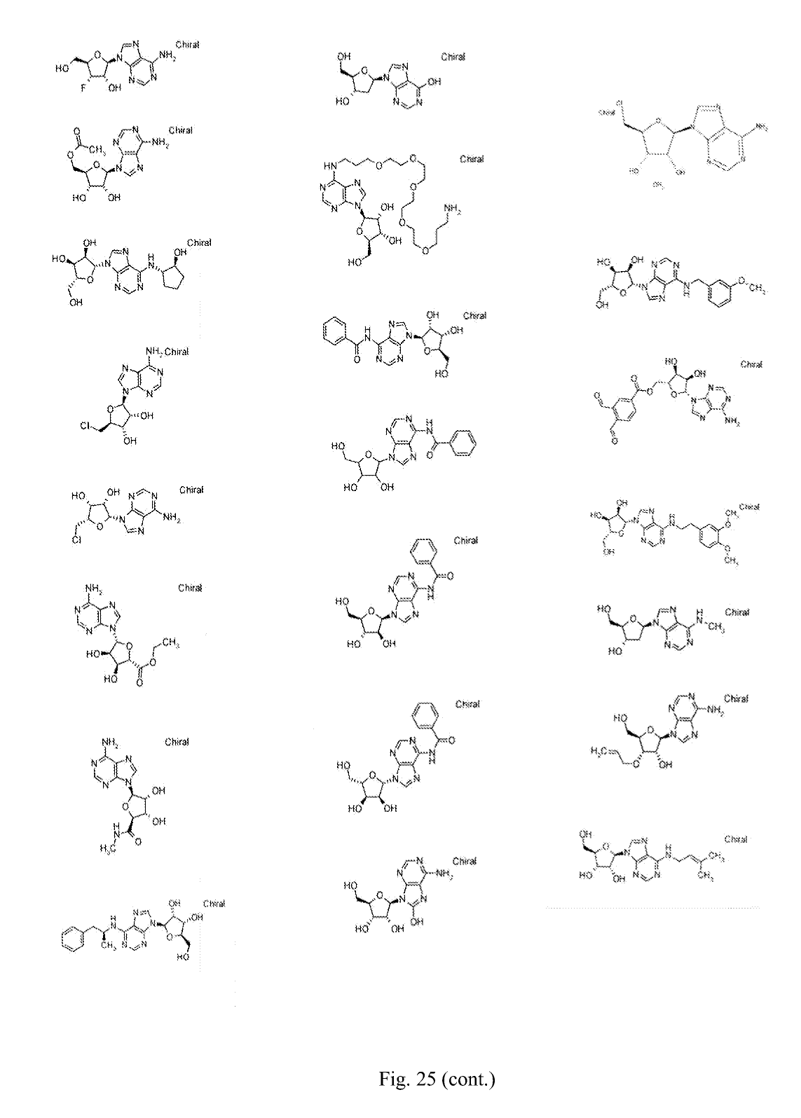

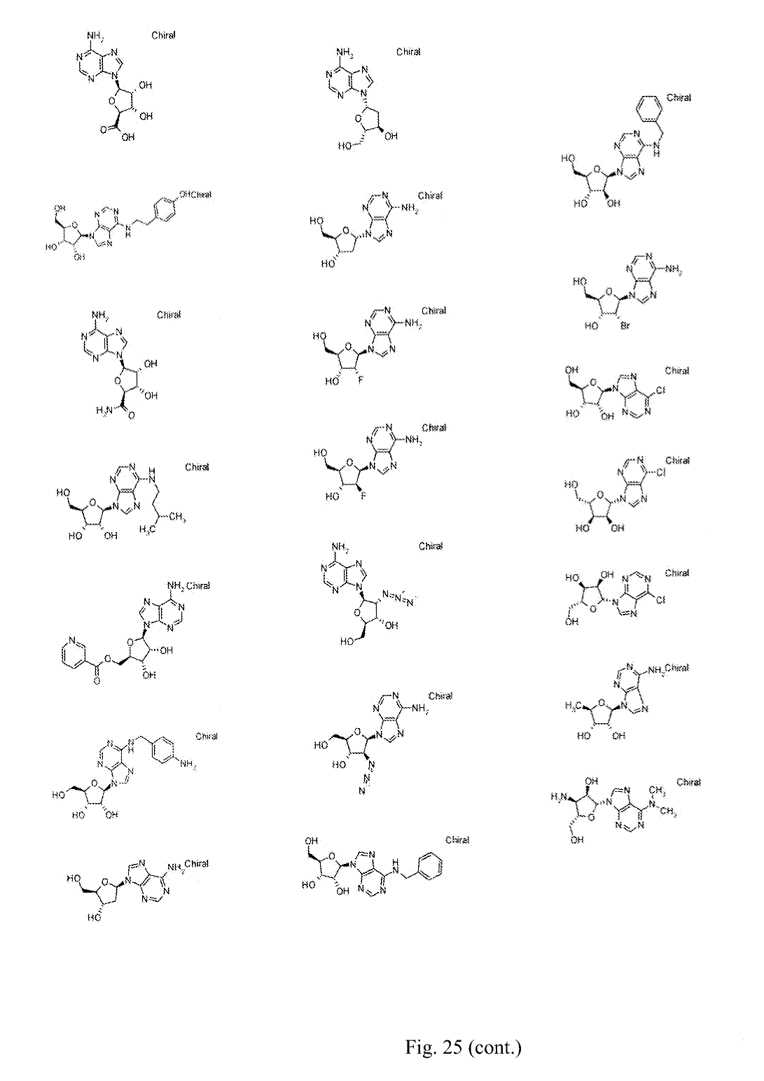

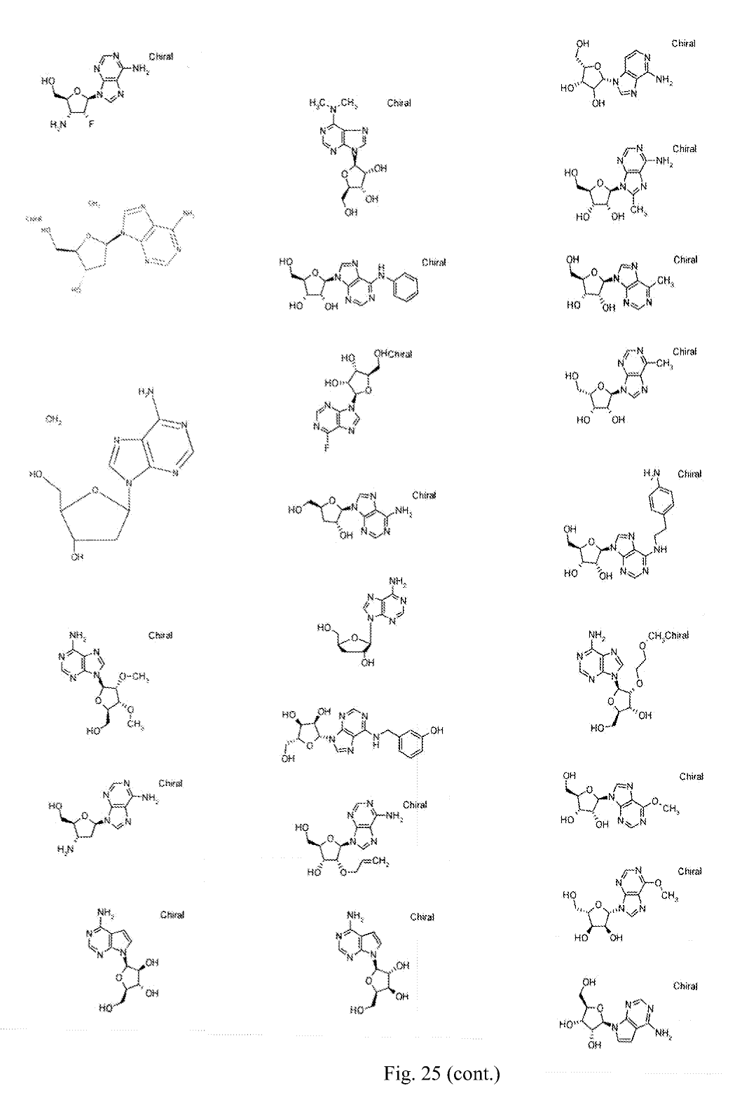

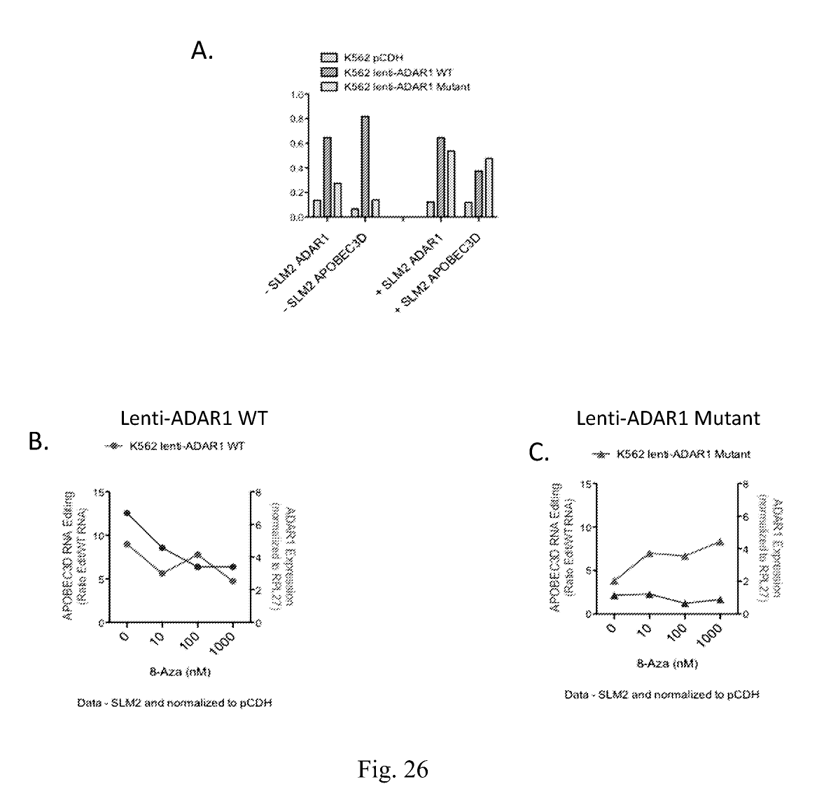

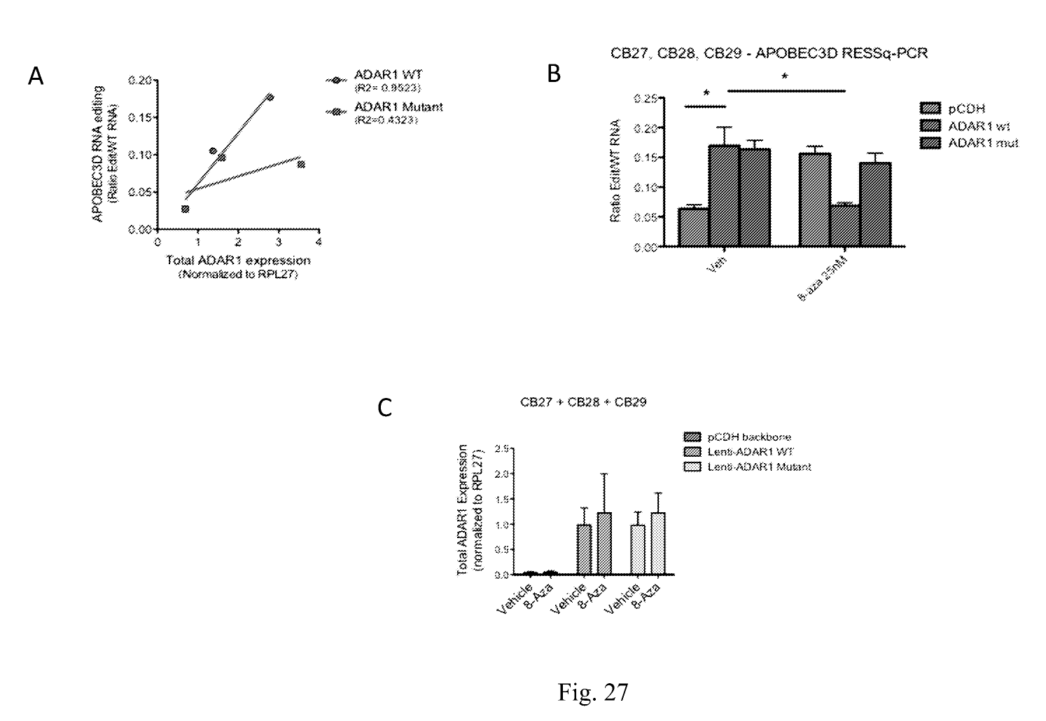

As proof-of-concept that small molecule inhibitors could block RNA editing activity, CD34⁺ cells from a CML blast crisis patient were treated with vehicle or 1 μM 8-azaadenosine (Veliz et al., JACS 2003) for 24 hours and co-cultured on humanized bone marrow stromal cells (SL/M2) for 5 days. Direct sequencing of two CSC-specific RNA editing biomarkers in APOBEC3D showed a reduction in edit/wild-type sequence ratios following 24 hrs of 8-azaadenosine treatment (FIGS. 21, 26, 27). These data show that RESSq-PCR for these and other RNA editing biomarkers can form the basis of screening methods to test activity of RNA editing inhibitor compounds derived from 8-azaadenosine and related small molecules (FIG. 25 and EP0066918) to identify novel agonists and inhibitors of RNA editing in high-throughput RESSq-PCR and RNA editing reporter screens. Thus, RESSq-PCR and RNA editing inhibitors is an allele-specific diagnostic assay that can be used for detection of primate-specific RNA editing events in cancer stem cells.

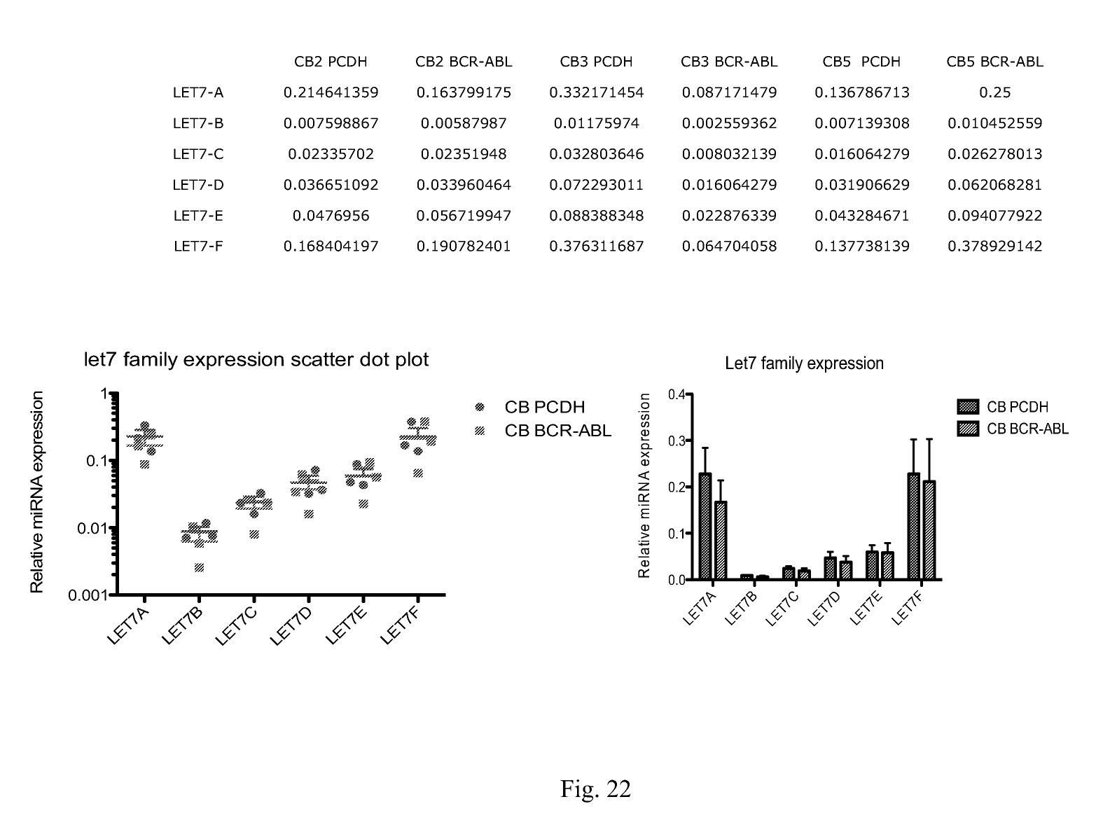

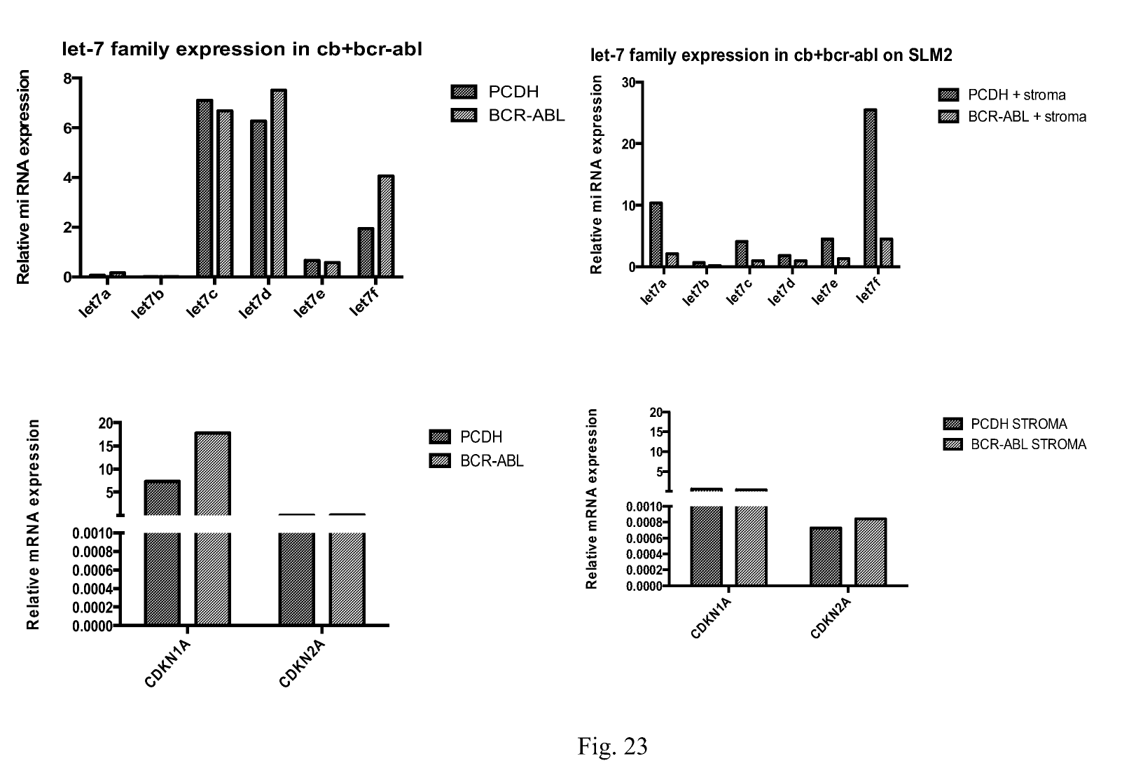

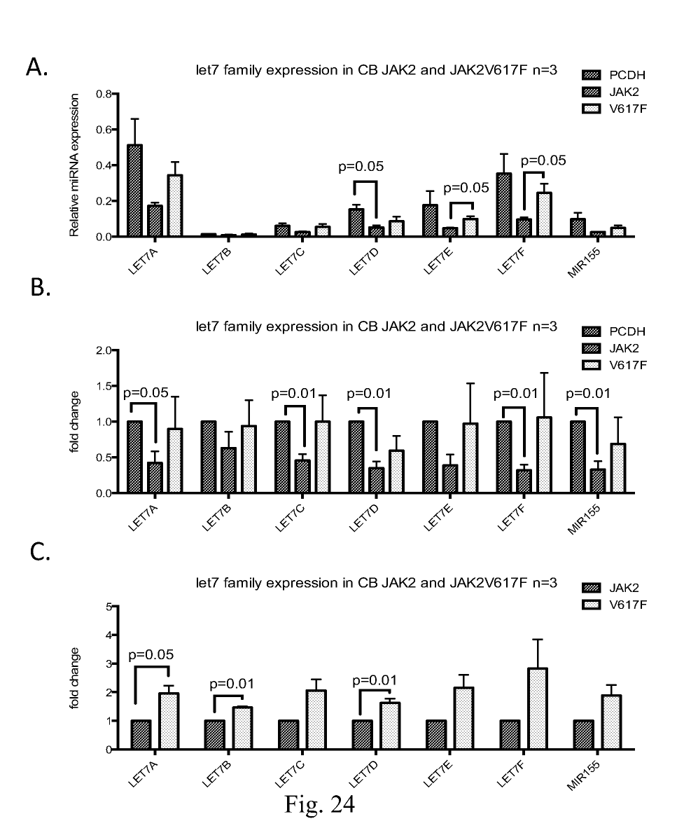

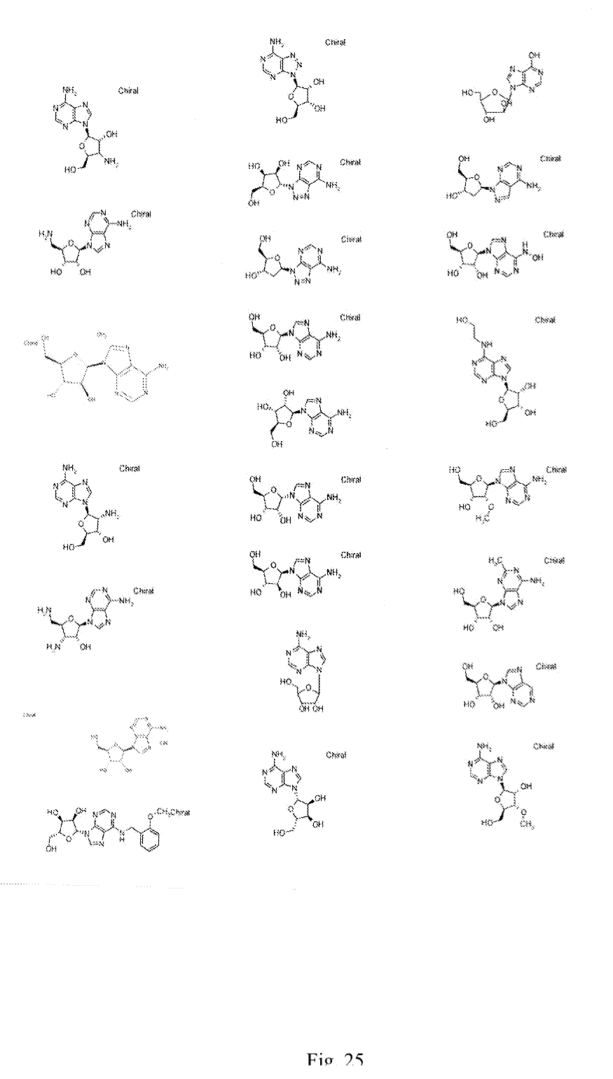

We have also demonstrated that let7 regulation by BCR-ABL and ADAR editing of miRs is niche dependent (FIGS. 22-23) and that JAK2 overexpression regulated let7 family members (FIG. 24). The data shown in FIG. 22 was obtained by transducing CD34⁺ CB n=3 with lenti-BCR-ABL and expression of let7 family was evaluated by RT-qPCR. Lentiviral over-expression of BCR-ABL in CB n=3 do not affect expression of members of let7 family.

### Generating and Manipulating Nucleic Acids:

In alternative embodiments, nucleic acids of the invention are made, isolated and/or manipulated by, e.g., cloning and expression of cDNA libraries, amplification of message or genomic DNA by PCR, and the like.

The nucleic acids used to practice this invention, whether RNA, iRNA, shRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including e.g. bacterial, fungal, mammalian, yeast, insect or plant cell expression systems.

Alternatively, nucleic acids used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68: 109; Beaucage (1981) Tetra. Lett. 22: 1859; U.S. Pat. No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones.

Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P I artificial chromosomes, see, e.g., Woon (1998) Genomics 50:306-316; P 1-derived vectors (PACs), see, e.g., Kern (1997) Biotechniques 23: 120-124; cosmids, recombinant viruses, phages or plasmids.

Nucleic acids or nucleic acid sequences used to practice this invention can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA, shRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Compounds use to practice this invention include nucleic acids, i.e., oligonucleotides, containing known analogues of natural nucleotides. Compounds use to practice this invention include nucleic-acid-like structures with synthetic backbones, see e.g., Mata (1997) Toxicol. Appl. Pharmacol. 144: 189-197; Strauss-Soukup (1997) Biochemistry 36:8692-8698; Samstag (1996) Antisense Nucleic Acid Drug Dev 6: 153-156. Compounds use to practice this invention include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, compounds used to practice this invention include genes or any segment of DNA or RNA involved in producing a polypeptide chain; it can include regions preceding and following the coding region (leader and trailer) as well as, where applicable, intervening sequences (introns) between individual coding segments (exons). “Operably linked” can refer to a functional relationship between two or more nucleic acid (e.g., DNA or RNA) segments. In alternative aspects, it can refer to the functional relationship of transcriptional regulatory sequence to a transcribed sequence. For example, a promoter can be operably linked to a coding sequence, such as a nucleic acid used to practice this invention, if it stimulates or modulates the transcription of the coding sequence in an appropriate host cell or other expression system. In alternative aspects, promoter transcriptional regulatory sequences can be operably linked to a transcribed sequence where they can be physically contiguous to the transcribed sequence, i.e., they can be cis-acting. In alternative aspects, transcriptional regulatory sequences, such as enhancers, need not be physically contiguous or located in close proximity to the coding sequences whose transcription they enhance.

In alternative aspects, the invention comprises use of “expression cassettes” comprising a nucleotide sequence used to practice this invention, which can be capable of affecting expression of the nucleic acid, e.g., a structural gene or a transcript (e.g., encoding a DRP or antibody) in a host compatible with such sequences. Expression cassettes can include at least a promoter operably linked with the polypeptide coding sequence or inhibitory sequence; and, in one aspect, with other sequences, e.g., transcription termination signals. Additional factors necessary or helpful in effecting expression may also be used, e.g., enhancers.

In alternative aspects, expression cassettes used to practice this invention also include plasmids, expression vectors, recombinant viruses, any form of recombinant “naked DNA” vector, and the like. In alternative aspects, a “vector” used to practice this invention can comprise a nucleic acid that can infect, transfect, transiently or permanently transduce a cell. In alternative aspects, a vector used to practice this invention can be a naked nucleic acid, or a nucleic acid complexed with protein or lipid. In alternative aspects, vectors used to practice this invention can comprise viral or bacterial nucleic acids and/or proteins, and/or membranes (e.g., a cell membrane, a viral lipid envelope, etc.). In alternative aspects, vectors used to practice this invention can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice this invention can be stably replicated by the cells during mitosis as an autonomous structure, or can be incorporated within the host's genome.

In alternative aspects, “promoters” used to practice this invention include all sequences capable of driving transcription of a coding sequence in a cell, e.g., a mammalian cell such as a brain cell. Thus, promoters used in the constructs of the invention include cis-acting transcriptional control elements and regulatory sequences that are involved in regulating or modulating the timing and/or rate of transcription of a gene. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

“Constitutive” promoters used to practice this invention can be those that drive expression continuously under most environmental conditions and states of development or cell differentiation. “Inducible” or “regulatable” promoters used to practice this invention can direct expression of the nucleic acid of the invention under the influence of environmental conditions or developmental conditions.

Cloning and transduction of pCDH vectors is described in the user manual for pCDH cDNA Cloning and Expression Lentivectors from System Biosystems, and which is incorporated herein by reference.

Methods related to ADAR1 as it relates to cancer stem cells, including full transcriptome RNA sequencing, assays and the like are disclosed in WO 2013/036867 and Jiang et al., Proc. Natl Acad Sci USA 2013, 110(3):1041-1046, which are incorporated herein by reference.

RNA Editing Site-Specific qPCR is described in Ye S, Dhillon S, Ke X, Collins A R & Day I N (2001) An efficient procedure for genotyping single nucleotide polymorphisms. Nucleic Acids Res 29, E88, and Chen et al., Anal Biochem, 2008, 375(1):46-52. Other methods for determining RNA editing sites are disclosed in, for example, Chateigner-Bouting and Small, Nucl Acids Res, 2007, 35(17), e114 and Paz et al., Genome Res., 2007, 17(11):1586-1595.

### Kits and Instructions:

The invention provides kits comprising compositions and/or instructions for practicing methods of the invention. As such, kits, cells, vectors, primers, and the like can also be provided. In alternative embodiments, the invention provides kits comprising: a composition used to practice a method of any of the invention, or a composition, a pharmaceutical composition or a formulation of the invention, and optionally comprising instructions for use thereof.

### Detailed Methods

### Primary Samples and Tissue Processing:

A large collection of leukemia patient samples and normal age-matched control bone marrow samples were obtained from consenting patients. Peripheral blood or bone marrow samples were processed by Ficoll density centrifugation and viable cells stored in liquid nitrogen. Normal peripheral blood mononuclear cells (MNC) were obtained from AllCells (Alameda, Calif.). Mononuclear cells from normal or CML patients were then further purified by magnetic bead separation of CD34⁺ cells (MACS; Miltenyi, Bergisch Gladbach, Germany) for subsequent FACS-purification of hematopoietic progenitor cells (CD34⁺CD38⁺Lin⁻) that represent the LSC fraction in BC CML (Jamieson et al.). Datasets from previous RNA-seq analyses of purified CML LSC are available through the NIH Sequence Read Archive (SRA), accession ID SRP028528.

### Primary CSC Purification:

For primary patient-derived LSC purification, CD34-selected cells were stained with fluorescent antibodies against human CD34, CD38, lineage markers (cocktail, all antibodies from BD Biosciences, San Diego, Calif.) and propidium iodide as previously described (Jiang et al., Jamieson et al, and Abrahamsson et al.). Following staining, cells were analyzed and sorted using a FACS Aria II, and hematopoietic progenitor (CD34⁺CD38⁺Lin⁻) populations were isolated. Freshly-sorted cells were collected in lysis buffer (Qiagen, Germantown, Md.) for RNA extraction followed by RNA-seq or qRT-PCR analyses as previously described (Jiang et al.).

### High-Fidelity PCR and Sanger Sequencing Analysis:

For PCR and targeted Sanger sequencing analysis, 1-2 μL of first-strand cDNA templates were prepared for PCR in 25-50 μL reaction volumes using the high-fidelity KOD Hot Start DNA Polymerase kit according to the manufacturer's instructions (EMD Millipore, Temecula, Calif.). “Outer” primers (Additional file 2: Table S2) used for sequencing produce predicted amplicons of approximately 150-250 nucleotides in length, and flank each editing site with approximately 50-100 bp on either side of the editing site to facilitate successful sequencing analysis. PCR cycling conditions were as follows: 95° C. for 2 minutes, followed by 35 cycles of 95° C. for 20 seconds, 62° C. for 10 seconds and 70° C. for 10 seconds, with a final extension step of 70° C. for 30 seconds. Production of amplicons of the predicted size was verified for each outer primer set by DNA gel electrophoresis using 10-20 μL of the completed reaction mixture separated on 2% agarose gels containing ethidium bromide and visualized under UV light. Then, 15 μL of each reaction was processed within 24 hrs for PCR purification and sequencing was performed on ABI 3730xl DNA Sequencers (Eton Bioscience, San Diego, Calif.). Sanger sequencing was carried out using the reverse outer primer used for PCR amplification, so edited loci are identified in the reverse complementary sequence as T/C nucleotides, except in cases where the gene products are transcribed from the reverse strand. Sequence chromatograms were analyzed using 4Peaks (by A. Griekspoor and Tom Groothuis) and peak heights calculated using ImageJ (NIH). For RNA editing analysis of sequencing chromatograms, ratios of edited/WT peaks were calculated using the raw peak amplitude of each sequence trace.

### Cell Lines and Culture Conditions:

K562 cells (ATCC, Manassas, Va.) were maintained in complete medium containing DMEM (Life Technologies, Carlsbad, Calif.), 10% fetal bovine serum (FBS), 1% Glutamax (Life Technologies), and 1% penicillin-streptomycin (Life Technologies). Parental cell lines and stably-transduced lines were authenticated as K562 by routine qRT-PCR analysis of BCR-ABL transcript levels (Jiang et al.). Mouse bone marrow stromal cell lines (SL and M2) expressing human interleukin-3 (IL-3), stem cell factor (SCF) and granulocyte-colony stimulating factor (G-CSF), which support erythroid and myeloid cell expansion and differentiation, were maintained under standard culture conditions, as previously described (Hogge et al.). Briefly, SL cells were grown in complete medium containing DMEM, 10% FBS, 1% Glutamax, and 1% penicillin-streptomycin, while M2 cells were grown in complete medium containing RPMI, 10% FBS, 1% Glutamax, and 1% penicillin-streptomycin (all from Life Technologies). Every four passages, cells were selected by addition of G418 and hygromycin to the culture media for one passage (3-4 days), to maintain human cytokine expression (Hogge et al.). All cell lines were maintained in T-25 or T-75 culture flasks and were passaged at dilutions of 1:5-1:10 every 2-4 days. Low passage aliquots of cells were thawed every two months to ensure consistency of experiments.

### Lentiviral Vector Preparation and ADAR1 Site-Directed Mutagenesis:

We have previously characterized lentiviral vectors (Thermo Scientific) for overexpression of human ADAR1 p150-IRES-GFP (Jiang et al.). For production of the catalytically-inactive ADAR1 mutated (ADAR1m) lentivirus, site-directed mutagenesis was carried out using the QuikChange II Site-Directed Mutagenesis Kit (Agilent) according to manufacturer's instruction. Mutagenic primers were designed to produce a nucleotide substitution of A5293C, which generates an E912A amino acid change and abolishes RNA editase activity (Lai et al.). Primers contained the desired mutation and anneal to the same sequence on opposite strands of the plasmid [(FW 5′-GTCAATGACTGCCATGCAGCAATAATCTCCCGG-3′ (SEQ ID NO:53), REV 5′-CCGGGAGATTATTGCTGCATGGCAGTCATTGAC-3′ (SEQ ID NO:54)]. XLI super competent cells were transformed with amplification products, after digestion with DpnI. Colonies were screened to identify mutated clones by DNA sequencing (Sanger sequencing, Eton Bioscience). Lentiviruses including control vectors (ORF) were produced according to established methods (Goff et al.) with some batches of lentivirus being produced by the GT3 Viral Vector Core Facility (UCSD). We have previously validated lentivirus transduction efficiency in normal cord blood, 293T cells and K562 cells, with an increase of approximately five-fold overexpression of ADAR1 transcripts confirmed by qRT-PCR analysis (Jiang et al).

Transduction of Human Cell Lines and Primary Cells with Lentiviral-ADAR1:

For preparation of stably-transduced K562 cell lines, 50,000 wild-type (wt) K562 cells were plated into 96-well U-bottom plates in complete culture medium and transduced with lentiviral vectors expressing GFP (ORF), ADAR1-GFP, or ADAR1m-GFP at multiplicities of infection (MOI) from 50-200. After transduction, cultures were expanded for at least 5 passages and then processed for FACS purification of GFP-positive cells to establish pure stably-transduced lines. Stable expression of lentivirus-enforced ADAR1 conferring increased transcript levels of human ADAR1 in K562-ADAR1 cells was confirmed at every 5 passages by qRT-PCR.

For transduction of human normal HSC and CML progenitors, 50,000 CD34-selected (MACS, Miltenyi, Auburn, Calif.) cells were plated in 96-well U-bottom plates in StemPro media (Life Technologies) supplemented with human cytokines (IL-6 10 ng/mL, FLT3 50 ng/mL, SCF 50 ng/mL, and Tpo 10 ng/mL) as previously described Jiang et. al and Abrahamsson et al. Twenty-four hours later, cells were transduced with lentiviral vectors (ADAR1 or ORF control, MOI=50-100) for up to five days. For co-culture experiments, CD34-selected CP CML cells were transferred three days after transduction (MOI=75) to monolayers of mouse bone marrow stromal cell cultures containing a 1:1 mixture of irradiated SL and M2 cells (50,000 total stromal cells per well in 24-well plates) (Goff et al.). Primary transduced cells were maintained in co-culture for 5-days in Myelocult (Stem Cell Technologies, Vancouver, Canada) and then the total culture was harvested in lysis buffer for RNA extraction and qRT-PCR and RESSq-PCR analyses.

### Generation of a Stable ADAR1 RNA Editing Detection Model System:

For purification of stably-transduced K562 cell lines, K562 cells transduced with lentiviral-ADAR1 or ORF controls (MOI=50-200) were collected (minimum 1×10⁶ cells), washed in HBSS containing 2% FBS (staining media), and sorted using a FACS Aria II for high GFP signal to purify the highly-transduced cell population. Purified cells were collected in complete media and maintained under routine culture conditions for K562 cells. The lentiviral-ORF and ADAR1 vectors include a blasticidin-resistance gene, but we observed no significant change in ADAR1 expression in our stably-transduced cell lines following selection with blasticidin, and therefore no subsequent selection method was used after FACS purification.

### Nucleic Acid Isolation, Reverse Transcription and Quantitative RT-PCR:

Cell lines, lentivirus-transduced primary hematopoietic cells, or FACS-purified primary cells were harvested in lysis buffer (Qiagen). RNA was purified using RNeasy extraction kits with a DNase (Qiagen) incubation step to digest any trace genomic DNA present. For RNA extraction from cell line lysates, 1-2×10⁶ cells were extracted using RNeasy mini columns, and for primary cells, 5-10×10⁴ cells were lysed and extracted using RNeasy micro columns. Genomic DNA was purified from equal numbers of cells lysed separately using the QIAamp DNA Blood Mini Kit (Qiagen) including an RNase A incubation step to digest any RNA present (Qiagen). RNA was stored at −80° C. and gDNA stored at −20° C. Immediately prior to reverse transcription of RNA samples, nucleic acid concentrations were quantified on a NanoDrop 2000 spectrophotometer (Thermo Scientific), and purity was considered acceptable if A260/A280 values were >1.8. For standard qRT-PCR analysis of relative mRNA expression levels, DNA was synthesized using 50 ng-1 μg of template RNA in 20 μL reaction volumes using the First-Strand SuperScript III Reverse Transcriptase Supermix (Life Technologies) followed by incubation with RNase H according to the manufacturer's protocol and as described previously (Abrahamsson et al.). All cDNA products were stored at −20° C.

Because RNA editing events often occur in pre-processed RNA species, for cDNA preparation, we tested three different conditions, including (1) reverse transcription with gene-specific primers, (2) random hexamer primers only, or (3) a supermix containing both random hexamers and oligo-dT primers. Using cDNA prepared with all three methods was suitable for detection of intronic regions in cDNA prepared from DNase-digested RNA extracts, and detected increased RNA editing in K562-ADAR1 cells. We therefore proceeded with the standard supermix reverse transcription method for RESSq-PCR, as this would provide the most versatility for use of valuable human tissue samples and would allow analysis of total mRNA expression of other genes in the same samples.

Primers were synthesized by ValueGene (San Diego, Calif.) and diluted to 10 μM working dilutions in DNase/RNase-free water. qRT-PCR was performed in duplicate using cDNA (1 μL reverse transcription product per reaction) on an iCycler (Bio-Rad, Hercules, Calif.) using SYBR GreenER Super Mix (Life Technologies) in 25-μL volume reactions containing 0.2 μM of each forward and reverse primer. Cycling conditions were as follows: 50° C. for 2 minutes, then 95° C. for 8 minutes and 30 seconds, followed by 40 cycles of 95° C. for 15 seconds and 60° C. for 60 seconds. Melting curve analysis was performed on each plate according to the manufacturer's instructions. For standard qRT-PCR, HPRT mRNA transcript levels were used to normalize Ct values obtained for each gene, and relative expression levels were calculated using the 2^{-ddCt} method. To ensure validity of results, only Ct values <35 were used in gene expression analyses. All primer sets were tested in a no-template control (NTC) reaction containing only water instead of cDNA, and all gave Ct values >35 in NTC reactions. Production of a single amplicon of the expected size was verified for each primer set by DNA gel electrophoresis on 2% agarose gels containing ethidium bromide. For all cell line experiments, assays were repeated at least three times using separate RNA extracts and cDNA preparations.

### RNA Editing Fingerprint Assay:

In order to implement a rapid, cost-effective and clinically amenable method to detect a CSC-specific RNA editing fingerprint of cancer progression, we devised an RNA editing site-specific primer design strategy that is compatible with SYBR green qRT-PCR protocols (RESSq-PCR). Since RNA-edited transcripts are predicted to differ from wild-type (WT) sequences at only one nucleotide position, detection of RNA editing by qRT-PCR requires highly sensitive and selective primer design strategies. We have previously developed qRT-PCR primers that specifically recognize a gene product with a single point mutation (JAK2 V617F (Geron et al.), and here we employed a similar approach in designing RESSq-PCR primers. Allele-specific PCR strategies, based on positioning the 3′ base of a PCR primer to match one variant allele, have been used for the detection of SNPs and mutations in human genomic DNA or cDNA Ye et al., however are not routinely used in quantitative detection of RNA single nucleotide modifications.

The RESSq-PCR assay primer design was applied to specific cancer and stem cell-associated loci (FIG. 14). Efficiency of all primer sets (FIG. 16 and Table was tested using serial dilutions of K562-ADAR1 cDNA. Primer sets were tested experimentally for human specificity and were considered to be human-specific if they returned Ct values >35 in cDNA prepared from mouse bone marrow stromal cell controls. Editing site-specific primers for some loci (FIG. 14) either failed to discriminate between cDNA and gDNA, or K562-ADAR1 cells did not display increased editing by Sanger sequencing, and therefore were not continued for assay development. RESSq-PCR was performed in duplicate using cDNA (1-5 μL reverse transcription product per reaction) or gDNA (10-200 ng input gDNA) on an iCycler (Bio-Rad) using SYBR GreenER Super Mix (Life Technologies) in 25-μL volume reactions containing 0.2 μM of each forward and reverse primer. Cycling conditions were the same as for standard qRT-PCR. Relative RNA editing rates (Relative edit/WT RNA) were calculated using the following calculation: 2^{-(Ct Edit-Ct WT)}.

### Statistical Methods:

qRT-PCR data were measured as a continuous outcome and each group was assessed for distribution. For normally distributed data, the Student's t-test was applied to compare differences in RNA expression and editing ratios calculated by Sanger sequencing and RESSq-PCR, and values are expressed as means±SEM. Experiments were performed in triplicate on blind-coded samples, and all statistical analyses were performed using GraphPad Prism (San Diego, Calif.).

### REFERENCES

Jiang Q, Crews L A, Barrett C L, Chun H J, Court A C, Isquith J M, Zipeto M A, Goff D J, Minden M, Sadarangani A, et al: ADAR1 promotes malignant progenitor reprogramming in chronic myeloid leukemia. Proc Natl Acad Sci USA 2013, 110:1041-1046.
Jamieson C H, Ailles L E, Dylla S J, Muijtjens M, Jones C, Zehnder J L, Gotlib J, Li K, Manz M G, Keating A, et al: Granulocyte-macrophage progenitors as candidate leukemic stem cells in blast-crisis CML. N Engl J Med 2004, 351:657-667.
Abrahamsson A E, Geron I, Gotlib J, Dao K H, Barroga C F, Newton I G, Giles F J, Durocher J, Creusot R S, Karimi M, et al: Glycogen synthase kinase 3beta missplicing contributes to leukemia stem cell generation. Proc Natl Acad Sci USA 2009, 106:3925-3929.
Hogge D E, Lansdorp P M, Reid D, Gerhard B, Eaves C J: Enhanced detection, maintenance, and differentiation of primitive human hematopoietic cells in cultures containing murine fibroblasts engineered to produce human steel factor, interleukin-3, and granulocyte colonystimulating factor. Blood 1996, 88:3765-3773.
Gommans W M, McCane J, Nacarelli G S, Maas S. A mammalian reporter system for fast and quantitative detection of intracellular A-to-I RNA editing levels. Analytical Biochemistry. 2010; 399(2):230-6. Epub Jan. 7, 2010
Goff D J, Recart A C, Sadarangani A, Chun H J, Barrett C L, Krajewska M, Leu H, Low-Marchelli J, Ma W, Shih A Y, et al: A Pan-BCL2 inhibitor renders bone-marrow-resident human leukemia stem cells sensitive to tyrosine kinase inhibition. Cell Stem Cell 2013, 12:316-328.
Liu H, Liu Y, Liu Q F. [Progress of study on microRNA and chronic myeloid leukemia]. Zhongguo shi yan xue ye xue za zhi/Zhongguo bing li sheng li xue hui=Journal of experimental hematology/Chinese Association of Pathophysiology. 2012; 20(1):192-5. Epub Mar. 7, 2012.
Lai F, Drakas R, Nishikura K: Mutagenic analysis of double-stranded RNA adenosine deaminase, a candidate enzyme for RNA editing of glutamategated ion channel transcripts. J Biol Chem 1995, 270:17098-17105.
Geron I, Abrahamsson A E, Barroga C F, Kavalerchik E, Gotlib J, Hood J D, Durocher J, Mak C C, Noronha G, Soll R M, et al: Selective inhibition of JAK2-driven erythroid differentiation of polycythemia vera progenitors. Cancer Cell 2008, 13:321-330.
Agirre X, Jimenez-Velasco A, San Jose-Eneriz E, Garate L, Bandres E, Cordeu L, et al. Down-regulation of hsa-miR-10a in chronic myeloid leukemia CD34+ cells increases USF2-mediated cell growth. Molecular cancer research: MCR. 2008; 6(12):1830-40. Epub 2008/12/17.
Rokah O H, Granot G, Ovcharenko A, Modai S, Pasmanik-Chor M, Toren A, et al. Downregulation of miR-31, miR-155, and miR-564 in chronic myeloid leukemia cells. PloS one. 2012; 7(4):e35501. Epub 2012/04/19.
Tomura M, Sakaue-Sawano A, Mori Y, Takase-Utsugi M, Hata A, Ohtawa K, Kanagawa O, Miyawaki A. (2013) Contrasting quiescent G0 phase with mitotic cell cycling in the mouse immune system. PLOS One. 8(9):e73801PMID: 24066072
Sakaue-Sawano A, Kurokawa H, Morimura T, Hanyu A, Hama H, Osawa H, Kashiwagi S, Fukami K, Miyata T, Miyoshi H, Imamura T, Ogawa M, Masai H, Miyawaki A. (2008) Visualizing spatiotemporal dynamics of multicellular cell-cycle progression. Cell. February 8; 132(3):487-98. PMID: 18267078
Veliz E A, Easterwood L M & Beal P A (2003) Substrate analogues for an RNA-editing adenosine deaminase: mechanistic investigation and inhibitor design. J Am Chem Soc 125, 10867-10876.
Ye S, Dhillon S, Ke X, Collins A R & Day I N (2001) An efficient procedure for genotyping single nucleotide polymorphisms. Nucleic Acids Res 29, E88.

A number of embodiments of the invention have been described. Nevertheless, it will be understood that various modifications may be made without departing from the spirit and scope of the invention. Accordingly, other embodiments are within the scope of the following claims.

1.-21. (canceled)

22. A method of measuring and tracing the A-to-I RNA editing changes in a cancer stem cell(s) comprising
a) introducing into cancer stem cells a reporter vector comprising dual-luciferase, or enhanced green fluorescence protein (EGFP) or enhanced yellow fluorescence protein (EYFP) as a readout of A-to-I editing activity and a stop codon (TAG) in a hairpin structure which is part of the promoter sequence, wherein the stop codon is removed due to RNA editing resulting in a reporter gene signal as a readout of RNA editing level in which increased reporter activity correlates with higher RNA-editing activity and decreased reporter activity correlates with lower RNA-editing activity; and
b) correlating changes in A-to-I RNA editing after exposure to agents.

23. The method of claim 22 wherein the agents are toxins, other chemicals or biological agents.

24. The method of claim 22, wherein the cancer stem cell(s) are in an in vitro stromal co-culture system or in vivo xenograft mouse model.

25.-33. (canceled)

34. A method for treating, ameliorating or preventing diseases and conditions associated with the downregulation of one or more microRNA identified in FIGS. 8-11, comprising: administering to a subject in need of treatment a composition that results in upregulation of one or more of the downregulated microRNA, wherein the composition is one or more of antisense DNA, RNAi, ribozyme, short hairpin RNA (shRNA), a small molecule, an antibody or antibody fragment, a small HA oligosaccharide, or soluble HA-binding proteins.

35. The method according to claim 34, wherein the downregulation of the microRNA is associated with a stem cell.

36. The method according to claim 35, wherein the stem cell is a cancer stem cell.

37. The method according to claim 36, wherein the cancer stem cell is a leukemic stem cell.

38. The method according to claim 34, wherein the condition or disease is cancer or inflammatory disease.

39. The method according to claim 34, wherein the condition or disease is myeloproliferative neoplasm.

40. The method according to claim 34, wherein the condition or disease is chronic myeloid leukemia (CML) or acute myeloid leukemia (AML).

41. The method according to claim 34, wherein the condition or disease is CML that is in the blast phase.

42. The method according to claim 34, wherein the composition administered is an inhibitor of ADAR1.

43. A method for monitoring the success of treatment in a subject with cancer or an inflammatory disease comprising:
(a) isolating stem cells from the subject during or after treatment;
(b) processing the isolated stems cells to detect and/or quantitate one or more microRNA identified in FIGS. 8-11; and
(c) determining if the level of expression of the microRNA has decreased or increased from the levels of expression of the microRNA seen before treatment or as compared to normal controls;
wherein if the level of expression of one or more microRNA has decreased the subject should be treated or enrolled in a clinical trial.

44. The method according to claim 43, wherein the subject has been diagnosed with CML.

45. The method according to claim 34 wherein the composition upregulates Let7a.

46. The method of claim 45 wherein the composition is a vitamin D3 derivative.

47. (canceled)

48. A method for screening for agonists and inhibitors of RNA editing comprising:
(a) contacting a CD34⁺ cell which overexpresses ADAR1 with a test compound; and
(b) determining whether the test compound acts as an agonist or inhibitor of RNA editing as determined using a fluorescent A-to-I editing reporter vector which is transduced into the CD34⁺ cell of step (a); and
(c) measuring reporter gene activity.

49. The method of claim 48, wherein the reporter vector comprises dual-luciferase, or enhanced green fluorescence protein (EGFP) or enhanced yellow fluorescence protein (EYFP) as a readout of A-to-I editing activity and a stop codon (TAG) in a hairpin structure which is part of the promoter sequence, wherein the stop codon is removed due to RNA editing resulting in a reporter gene signal as a readout of RNA editing level in which increased reporter activity correlates with higher RNA-editing activity and decreased reporter activity correlates with lower RNA-editing activity.

50.-61. (canceled)

62. A method for detecting leukemic progression into blast phase comprising the steps of:
(a) collecting a blood sample from a patient with leukemia;
(b) isolating mononuclear cells from the blood sample;
(c) isolating CD34⁺ cells;
(d) isolating RNA from the CD34⁺ cells;
(e) generating cDNA from the isolated RNA;
(f) performing quantitative PCR using one or more single nucleotide variant site-specific primer sets for the genes MDM2, APOBEC3D, GLI1, AZIN1, SRPN, GSK3B, PTPN14, SF3B3, ABI1, or LYST; and
(g) quantifying the data to determine the relative A-to-G(I) editing ratios in one or more of MDM2, APOBEC3D, GLI1, AZIN1, SRPN, GSK3B, PTPN14, SF3B3, ABI1, LYST and MDM4, wherein if the editing ratio is higher than a normal standard or a previous determination from the same patient obtained in chronic phase the finding indicates the patient should be treated or entered into a clinical trial.

63. The method of claim 62, wherein the one or more primer sets are represented by SEQ ID Nos. 1-4 for MDM2 position 69237534 (chr 12); SEQ ID NOS. 5-8 for APOBEC3D position 39415872 (chr 22); SEQ ID NOS:9-12 for APOBEC3D position 39415911 (chr 22); SEQ ID NOS:13-16 for SRP9 position 225976198 (chr 1); SEQ ID NOS: 17-20 for Gli1 position 57864624 (chr 12); SEQ ID NOS:21-24 for Gli1 position 57864911(negative control); SEQ ID NOS:25-28 for GSK3B position 119545199 (chr 3); SEQ ID NOS:29-32 for AZIN position 103841636 (chr 8); SEQ ID NOS:33-36 for PTPN14 position 214529774 (chr 1); SEQ ID NOS:37-40 for SF3B3 position 70610885 (chr 16); SEQ ID NOS:41-44 for ABI1 position 27049636 (chr 10); SEQ ID NOS:45-48 for LYST position 235990569 (chr 1); and SEQ ID NOS:49-52 for MDM4 position 204521159 (chr 1).

64. A method for testing whether an agent is effective for reducing the editing activity of ADAR1 comprising:
(a) adding the agent to cells that have ADAR1 activity;
(b) isolating RNA from the cells of step (a);
(c) generating cDNA from the isolated RNA;
(d) performing quantitative PCR using one or more primer sets for the genes selected from the group consisting of MDM2, APOBEC3D, GLI1, AZIN1, SRPN, GSK3B, PTPN14, SF3B3, ABI1, and LYST; and
(e) quantifying the data to determine the relative A-to-G(I) editing ratios in one or more of MDM2, APOBEC3D, GLI1, AZIN1, SRPN, GSK3B, PTPN14, SF3B3, ABI1, LYST and MDM4, wherein if the ratio of edited/wild-type is lower than cells not treated with the tested agent indicates that the agent lowers the editing activity of ADAR1 and may be useful for treating conditions associated with high levels of ADAR1 activity.

65. The method of claim 64, wherein the one or more primer sets are represented by SEQ ID Nos. 1-4 for MDM2 position 69237534 (chr 12); SEQ ID NOS. 5-8 for APOBEC3D position 39415872 (chr 22); SEQ ID NOS:9-12 for APOBEC3D position 39415911 (chr 22); SEQ ID NOS:13-16 for SRP9 position 225976198 (chr 1); SEQ ID NOS: 17-20 for Gli1 position 57864624 (chr 12); SEQ ID NOS:21-24 for Gli1 position 57864911(negative control); SEQ ID NOS:25-28 for GSK3B position 119545199 (chr 3); SEQ ID NOS:29-32 for AZIN position 103841636 (chr 8); SEQ ID NOS:33-36 for PTPN14 position 214529774 (chr 1); SEQ ID NOS:37-40 for SF3B3 position 70610885 (chr 16); SEQ ID NOS:41-44 for ABI1 position 27049636 (chr 10); SEQ ID NOS:45-48 for LYST position 235990569 (chr 1); and SEQ ID NOS:49-52 for MDM4 position 204521159 (chr 1).

66. The method of claim 64, wherein the cells that have ADAR1 activity are cells containing a wild-type ADAR1 expression vector.

67. The method of claim 66, wherein the cells are K562 cells.

68. The method of claim 67, wherein the cells are stably-transduced with lentiviral-ADAR1.

69. The method of claim 64, wherein the cells in step (A) are CD34⁺ cells obtained from primary CIVIL patients; or cells obtained from an immunocompromised non-human animal transplanted with primary blast crisis cells.

70. The method of claim 64, wherein the cells in step (A) are treated with RNA editing inhibitory agents.

71. A method for detecting leukemic progression into blast phase comprising the steps of:
(a) collecting a blood sample from a patient with leukemia;
(b) isolating mononuclear cells from the blood sample;
(c) isolating CD34⁺ cells
(d) isolating RNA from the CD34⁺ cells;
(e) converting the RNA from step (d) into cDNA;
(f) evaluating miRNA expression using MiScript qPCR array; and
(g) determining if one or more of mir26a-5p, mir26b-5p, mir155-5P, mir21-5P, mir125a-5P, mir23b-3P, let7c, let7e, mir150-5p, or let7d are downregulated as compared to a normal control or a previous sample from the patient while in chronic phase, wherein if one or more of mir26a-5p, mir26b-5p, mir155-5P, mir21-5P, mir125a-5P, mir23b-3P, let7c, let7e, mir150-5p, or let7d are downregulated indicates that the patient is in or entering blast phase and should be treated or enrolled in a clinical trial.

72. A method for testing whether an agent is effective for reducing the editing activity of ADAR1 comprising:
(a) adding the agent to cells that have ADAR1 activity;
(b) isolating RNA from the cells of step (a);
(c) generating cDNA from the isolated RNA;
(d) converting the RNA from step (c) into cDNA;
(e) evaluating miRNA expression using MiScript qPCR array;
(f) determining if one or more of mir26a-5p, mir26b-5p, mir155-5P, mir21-5P, mir125a-5P, mir23b-3P, let7c, let7e, mir150-5p, or let7d are upregulated as compared to cells not treated with the tested agent, wherein if one or more of mir26a-5p, mir26b-5p, mir155-5P, mir21-5P, mir125a-5P, mir23b-3P, let7c, let7e, mir150-5p, or let7d are upregulated may indicate that the tested agent inhibits ADAR1 activity and may be useful to treat conditions associated with high ADAR1 editing activity.

73. The method of claim 72, wherein the cells that have ADAR1 activity are cells containing a wild-type ADAR1 expression vector.

74. The method of claim 72, wherein the cells are K562 cells.

75. The method of claim 74, wherein the cells are stably-transduced with lentiviral-ADAR1.

76. The method of claim 49, wherein the reporter vector further comprises an opposite oriented Alu-sequence to detect RNA editing in non-coding regions.

77. The method of claim 22, wherein the reporter vector further comprises an opposite oriented Alu-sequence to detect RNA editing in non-coding regions.

78. The method of claim 42, wherein the inhibitor of ADAR1 is 8-azaadenosine or an 8-azaadenosine derivative.
